SEQUENCE LISTING (35 sequences)

SEQ ID NO: 1 - DNA, 1581 bp, Canis familiaris
  CDS (3)..(305)
  repeat_unit (514)..(515): CT dinucleotide repeat polymorphism having 6, 14 or 17
agtccaaaaccagccccagtgtgttcccgctgagcctctgccaccaggagtcagaagggtacgtggtcatcggctgcctggtgcagggattcttcccaccggagcctgtgaacgtgacctggaatgccggcaaggacagcacatctgtcaagaacttcccccccatgaaggctgctaccggaagcctatacaccatgagcagccagttgaccctgccagccgcccagtgccctgatgactcgtctgtgaaatgccaagtgcagcatgcttccagccccagcaaggcagtgtctgtgccctgcaaaggtcagagggcaggctggggaggggcaggggccccacatcctcactctgaccctccacttggagttctggccccaaggacactccacggggaggacagtgggctgctgggctgagctcccagcaagtggccaaggtggggcctccatgaaggacctggagggtggcagggggcaggcaggcagagggtgcacactgacctgttccaatctcwgctcctgaagataacagtcatccgtgtcnnnatccatgtccctcgtgcaatgagccccgcctgtcactacagaagccagccctcgaggatctgcttttaggctccaatgccagcctcacatgcacactgagtggcctgaaagaccccaagggtgccaccttcacctggaacccctccaaagggaaggaacccatccagaagaatcctgagcgtgactcctgtggctgctacagtgtgtccagtgtcctaccaggctgtgctgatccatggaaccatggggacaccttctcctgcacagccacccaccctgaatccaagagcccgatcactgtcagcatcaccaaaaccacaggtgggcccagaccctgcccgtgaggcactgcttggcacacaaaagtttgtgaggcaactcctaagcctgcttccttcctctagcccctgggcttgggtgctcccacccacattttacaaagggaaactgtggcatggggtgctatggggaagaaggctcttcccccaccccagatccctgacctggctctctgtcctgcagagcacatcccgccccaggtccacctgctgccgccgccgtcggaagagctggccctcaatgagctggtgacactgacgtgcttggtgaggggcttcaaaccaaaagatgtgctcgtacgatggctgcaagggacccaggagctaccccaagagaagtacttgacctgggagcccctgaaggagcctgaccagaccaacatgtttgccgtgaccagcatgctgagggtgacagccgaagactggaagcagggggagaagttctcctgcatggtgggccacgaggctctgcccatgtccttcacccagaagaccatcgaccgcctggcgggtaaacccacccacgtcaacgtgtctgtggtcatggcagaggtggacggcatctgctactaaaccgcccaatcttccctccctaaataaactccatgcttgcccaaagcagccccgtgcttccatcaggccgcctgtctgtccatattcggggtctgtggcatactgaggcaggggtagagctc

SEQ ID NO: 2 - protein, 343 aa, Canis familiaris
SKTSPSVFPLSLCHQESEGYVVIGCLVQGFFPPEPVNVTWNAGKDSTSVKNFPPMKAATGSLYTMSSQLTLPAAQCPDDSSVKCQVQHASSPSKAVSVPCKDNSHPCHPCPSCNEPRLSLQKPALEDLLLGSNASLTCTLSGLKDPKGATFTWNPSKGKEPIQKNPERDSCGCYSVSSVLPGCADPWNHGDTFSCTATHPESKSPITVSITKTTEHIPPQVHLLPPPSEELALNELVTLTCLVRGFKPKDVLVRWLQGTQELPQEKYLTWEPLKEPDQTNMFAVTSMLRVTAEDWKQGEKFSCMVGHEALPMSFTQKTIDRLAGKPTHVNVSVVMAEVDGICY

SEQ ID NO: 3 - DNA, 542 bp, Canis familiaris
  CDS (2)..(199)
  variation (408)..(435): variable number of CT repeats [CT]n n = 12 or 14
tgtgaacgtgacctggaatgccggcaaggacagcacatctgtcaagaacttcccccccatgaaggctgctaccggaagcctatacaccatgagcagccagttgaccctgccagccgcccagtgccctgatgactcgtctgtgaaatgccaagtgcagcatgcttccagccccagcaaggcagtgtctgtgccctgcaaaggtcagagggcaggctggggaggggcaggggccccacatcctcactctgaccctccacttggagccgtgtccccaaggacactccacggggaggacagtgggctgctgggctgagctcccagcaagtggccaaggtggggcctccatgaaggacctggagggtggcagggggcaggcaggcagagggtgcacactgacctgttccaatctctctctctctctctctctctctctctgctcctgaagataacagtcatccgtgtcatccatgtccctcgtgcaatgagccccgcctgtcactacagaagccagccctcgaggatctgcttttaggctccaatgc

SEQ ID NO: 4 - protein, 98 aa, Canis familiaris
VNVTWNAGKDSTSVKNFPPMKAATGSLYTMSSQLTLPAAQCPDDSSVKCQVQHASSPSKAVSVPCKDNSHPCHPCPSCNEPRLSLQKPALEDLLLGSN

SEQ ID NO: 5 - DNA, 520 bp, Canis familiaris
  CDS (2)..(199)
  mutation (73)..(73): C or T
tgtgaacgtgacctggaatgccggcaaggacagcacatctgtcaagaacttcccccccatgaaggctgctactggaagcctatacaccatgagcagccagttgaccctgccagctgcccagtgccctgatgactcgtctgtgaaatgccaagtgcagcatgcttccagccccagcaaggcagtgtctgtgccctgcaaaggtcagagggcaggctggggaggggcaggggccccacatcctcactctgaccctccacttggagccgtgtccccaaggacactccacggggaggacagtgggctgctgggctgagctcccagcaagtggccaaggtggggcctccatgaaggacctggagggtggcagggggcaggcaggcagagggtgcacactgacctgttccaatctctctctctctgctcctgaagataactgtcctcatctgtgtccctcatgcaatgagccccacctgtcactacagaagccagccctcgaggatctgcttttaggctccaatgc

SEQ ID NO: 6 - protein, 96 aa, Canis familiaris
VNVTWNAGKDSTSVKNFPPMKAATGSLYTMSSQLTLPAAQCPDDSSVKCQVQHASSPSKAVSVPCKDNCPHLCPSCNEPHLSLQKPALEDLLLGSN

SEQ ID NO: 7 - DNA, 548 bp, Canis familiaris
  CDS (2)..(199)
  mutation (118)..(118): y = C or T
tgtgaacgtgacctggaatgccggcaaggacagcacatctgtcaagaacttcccccccatgaaggctgctactggaagcctatacaccatgagcagccagttgaccctgccagccgcycagtgccctgatgactcgtctgtgaaatgccaagtgcagcatgcttccagccccagcaaggcagtgtctgtgccctgcaaaggtcagagggcaggctggggaggggcaggggccccacatcctcactctgaccctccacttggagccgtgtccccaaggacactccacggggaggacagtgggctgctgggctgagctcccagcaagtggccaaggtggggcctccatgaaggacctggagggtggcagggggcaggcaggcagagggtgcacactgacctgttccaatctctctctctctctctctctctctctctctctcagctcctgaagataactgtcatccgtgtcatccatgtccctcgtgcaatgagccccgcctgtcactacagaagccagccctcgaggatctgcttttaggctccaatgc

SEQ ID NO: 8 - protein, 101 aa, Canis familiaris
VNVTWNAGKDSTSVKNFPPMKAATGSLYTMSSQLTLPAAQCPDDSSVKCQVQHASSPSKAVSVPCKAPEDNCHPCHPCPSCNEPRLSLQKPALEDLLLGSN

SEQ ID NO: 9 - DNA, 545 bp, Canis familiaris
  CDS (2)..(199)
  CDS (436)..(543)
tgtgaacgtgacctggaatgccggcaaggacagcacatctgtcaagaacttcccccccatgaaggctgctaccggaagcctatacaccatgagcagccagttgaccctgccagccgcccagtgccctgatgactcgtctgtgaaatgccaagtgcagcatgcttccagccccagcaaggcagtgtctgtgccctgcaaaggtcagagggcaggctggggaggggcaggggccccacatcctcactctgaccctccacttggagccgtgtccccaaggacactccacggggaggacagtgggctgctgggctgagctcccagcaagtggccaaggtggggcctccatgaaggacctggagggtggcagggggcaggcaggcagagggtgcacactgacctgttccaatctctctctctctctctctctctctctcagctcctgaagataactgtcatccgtgtcctcatccaagtccctcgtgcaatgagccccgcctgtcactacagaagccagccctcgaggatctgcttttaggctccaatgc

SEQ ID NO: 10 - protein, 102 aa, Canis familiaris
VNVTWNAGKDSTSVKNFPPMKAATGSLYTMSSQLTLPAAQCPDDSSVKCQVQHASSPSKAVSVPCKAPEDNCHPCPHPSPSCNEPRLSLQKPALEDLLLGSN

SEQ ID NO: 11 - DNA, 20 bp, Artificial Sequence
  Primer
tgtgaacgtgacctggaatg

SEQ ID NO: 12 - DNA, 20 bp, Artificial Sequence
  Primer
gcattggagcctaaaagcag

SEQ ID NO: 13 - DNA, 20 bp, Artificial Sequence
  Primer
tgtgccctgcaaagataaca

SEQ ID NO: 14 - DNA, 20 bp, Artificial Sequence
  Primer
agggctggcttctgtagtga

SEQ ID NO: 15 - DNA, 20 bp, Artificial Sequence
  Primer
cgtctgtgaaatgccaagtg

SEQ ID NO: 16 - DNA, 20 bp, Artificial Sequence
  Primer
agggctggcttctgtagtga

SEQ ID NO: 17 - DNA, 26 bp, Artificial Sequence
  Primer
gtcatccatgtccctcgtgcaatgag

SEQ ID NO: 18 - DNA, 1789 bp, Canis familiaris
agtgacctagcgtgtcattctgacccaggtctcggcatatgaactgcatgaccttgggctgtcactgaccatctctatgcagtttcctctagtgcaaagaaaaaatagccctcaccctgcctgtgaggccatgtaaggggtccagacagcactggcccaccagctcacagagtgtcctgtgtcacagagtccaaaaccagccccagtgtgttcccgctgagcctctgccaccaggagtcagaagggtacgtggtcatcggctgcctggtgcagggattcttcccaccggagcctgtgaacgtgacctggaatgccggcaaggacagcacatctgtcaagaacttcccccccatgaaggctgctaccggaagcctatacaccatgagcagccagttgaccctgccagccgcccagtgccctgatgactcgtctgtgaaatgccaagtgcagcatgcttccagccccagcaaggcagtgtctgtgccctgcaaaggtcagagggcaggctggggaggggcaggggccccacatcctcactctgaccctccacttggagttctggccccaaggacactccacggggaggacagtgggctgctgggctgagctcccagcaagtggccaaggtggggcctccatgaaggacctggagggtggcagggggcaggcaggcagagggtgcacactgacctgttccaatctctctctctctctctctctctctctctgctcctgaagataacagtcatccgtgtcatccatgtccctcgtgcaatgagccccgcctgtcactacagaagccagccctcgaggatctgcttttaggctccaatgccagcctcacatgcacactgagtggcctgaaagaccccaagggtgccaccttcacctggaacccctccaaagggaaggaacccatccagaagaatcctgagcgtgactcctgtggctgctacagtgtgtccagtgtcctaccaggctgtgctgatccatggaaccatggggacaccttctcctgcacagccacccaccctgaatccaagagcccgatcactgtcagcatcaccaaaaccacaggtgggcccagaccctgcccgtgaggcactgcttggcacacaaaagtttgtgaggcaactcctaagcctgcttccttcctctagcccctgggcttgggtgctcccacccacattttacaaagggaaactgtggcatggggtgctatggggaagaaggctcttcccccaccccagatccctgacctggctctctgtcctgcagagcacatcccgccccaggtccacctgctgccgccgccgtcggaagagctggccctcaatgagctggtgacactgacgtgcttggtgaggggcttcaaaccaaaagatgtgctcgtacgatggctgcaagggacccaggagctaccccaagagaagtacttgacctgggagcccctgaaggagcctgaccagaccaacatgtttgccgtgaccagcatgctgagggtgacagccgaagactggaagcagggggagaagttctcctgcatggtgggccacgaggctctgcccatgtccttcacccagaagaccatcgaccgcctggcgggtaaacccacccacgtcaacgtgtctgtggtcatggcagaggtggacggcatctgctactaaaccgcccaatcttccctccctaaataaactccatgcttgcccaaagcagccccgtgcttccatcaggccgcctgtctgtccatattcggggtctgtggcatactgaggcaggggtagagctc

SEQ ID NO: 19 - DNA, 21 bp, Artificial Sequence
  Primer
gagggtgcacactgacctgtt

SEQ ID NO: 20 - DNA, 19 bp, Artificial Sequence
  Primer
cacgagggacatggatgac

SEQ ID NO: 21 - DNA, 33 bp, Artificial Sequence
  Probe
ctctctctgctcctgaagataacagtcatccgt

SEQ ID NO: 22 - DNA, 23 bp, Artificial Sequence
  Primer
gcacactgacctgttccaatctc

SEQ ID NO: 23 - DNA, 21 bp, Artificial Sequence
  Primer
gggctggcttctgtagtgaca

SEQ ID NO: 24 - DNA, 30 bp, Artificial Sequence
  Probe
ataactgtcctcatctgtgtccctcatgca

SEQ ID NO: 25 - DNA, 21 bp, Artificial Sequence
  Primer
gagggtgcacactgacctgtt

SEQ ID NO: 26 - DNA, 19 bp, Artificial Sequence
  Primer
cacgagggacatggatgac

SEQ ID NO: 27 - DNA, 33 bp, Artificial Sequence
  Probe
ctctctcagctcctgaagataactgtcatccgt

SEQ ID NO: 28 - DNA, 26 bp, Artificial Sequence
  Primer
tctctctcagctcctgaagataactg

SEQ ID NO: 29 - DNA, 21 bp, Artificial Sequence
  Primer
gggctggcttctgtagtgaca

SEQ ID NO: 30 - DNA, 24 bp, Artificial Sequence
  Probe
ccgtgtcctcatccaagtccctcg

SEQ ID NO: 31 - DNA, 19 bp, Artificial Sequence
  Primer
tgcacactgacctgttcca

SEQ ID NO: 32 - DNA, 20 bp, Artificial Sequence
  Primer
gcattggagcctaaaagcag

SEQ ID NO: 33 - DNA, 38 bp, Artificial sequence
  Probe
ctctgctcctgaagataacagtcatccgtgtcatccat

SEQ ID NO: 34 - DNA, 38 bp, Artificial sequence
  Probe
gtgcaatgagccccgcctgtcactacagaagccagccc

SEQ ID NO: 35 - DNA, 530 bp, Canis familiaris
  CDS (2)..(199)
  mutation (73)..(73): y = C or T
tgtgaacgtgacctggaatgccggcaaggacagcacatctgtcaagaacttcccccccatgaaggctgctaytggaagcctatacaccatgagcagccagttgaccctgccagctgcycagtgccctgatgactcgtctgtgaaatgccaagtgcagcatgcttccagccccagcaaggcagtgtctgtgccctgcaaaggtcagagggcaggctggggaggggcaggggccccacatcctcactctgaccctccacttggagccgtgtccccaaggacactccacggggaggacagtgggctgctgggctgagctcccagcaagtggccaaggtggggcctccatgaaggacctggagggtggcagggggcaggcaggcagagggtgcacactgacctgttccaatctctctctctctctctctctctgctcctgaagataactgtcctcatctgtgtccctcatgcaatgagccccacctgtcactacagaagccagccctcgaggatctgcttttaggctccaatgc

(19) United States
(12) Patent Application Publication
(10) Pub. No.: US 2009/0028843 A1
(43) Pub. Date: Jan. 29, 2009

(54) IgA allelic variants

(76) Inventors: Chris Jones, Leicester (GB); Neale Fretwell, Leics (GB); Michael Day, Bristol (GB); Iain Peters, Bristol (GB)

Correspondence Address: Melissa W. Acosta;FULBRIGHT & JAWORSKI L.L.P., Suite 2800, 2200 Ross Avenue, Dallas, TX, 75201, US

(21) Appl. No.: 11/290,928

(22) Filed: Nov. 30, 2005

Related U.S. Application Data
(63) Continuation-in-part of application No. PCT/GB04/03527, filed on Aug. 13, 2004.
(60) Provisional application No. 60/631,644, filed on Nov. 30, 2004.

(30) Foreign Application Priority Data
Aug. 14, 2003 (GB) GB0319143.4
Nov. 18, 2003 (GB) GB0326848.9

(51) Int. Cl. A61K 39/395; G01N 33/563; G01N 33/53; C07H 21/00; A61K 38/16; A61K 31/145; A61K 36/886; A61P 37/00; G06F 7/20; A23K 1/00; G06F 17/30; A61K 31/202; A61K 31/12; A61K 31/375; C07K 16/00; A61K 31/716; A61K 31/195

(52) U.S. Cl. 424/130.1; 436/513; 436/501; 536/24.3; 530/387.1; 514/54; 514/563; 514/12; 514/474; 514/665; 514/683; 424/744; 514/560; 536/23.53; 707/104.1; 707/5; 426/2; 707/E17.044; 707/E17.014

(57) ABSTRACT

The invention relates to a method for determining susceptibility to an IgA-related disorder in an animal, the method comprising: a) identifying the or each IgA allelic variant present in a sample from the animal; and b) thereby determining whether the animal is susceptible to an IgA-related disorder.

### CROSS-REFERENCE TO RELATED APPLICATIONS

This application is a continuation-in-part of International Application No. PCT/GB04/03527 filed Aug. 13, 2004 that claims priority to United Kingdom Application No. 0319143.4 filed Aug. 14, 2003 and United Kingdom Application No. 0326848.9 filed Nov. 18, 2003. This application also claims priority to U.S. Provisional Application No. 60/631,644 filed Nov. 30, 2004. All applications are incorporated herein in their entirety.

### TECHNICAL FIELD

The present invention relates to the diagnosis and treatment of IgA-related disorders in animals, such as gastrointestinal, skin and respiratory disease, and to novel polynucleotides and polypeptides.

### BACKGROUND OF THE INVENTION

Dogs of the German shepherd breed are particularly susceptible to a number of inflammatory and immune-mediated alimentary diseases, including small intestinal bacterial overgrowth and inflammatory bowel disease (IBD). German shepherd dogs (GSD) have also been reported to have a relative IgA deficiency, on the basis of reduced concentrations of serum IgA as compared to control populations. Similar differences in IgA concentration have been reported in tears, duodenal juice, duodenal explant culture media and feces of this breed when compared with other breeds of dog. A reduced concentration of IgA has been found in the duodenal juice from GSD with small intestinal bacterial overgrowth compared with normal dogs, and tissue culture supernatants from twenty-four hour duodenal explants from GSD with chronic diarrhea produce less IgA than explants derived from affected dogs of other breeds. However, in the same populations there were no differences in the numbers of IgA⁺ plasma cells or CD4⁺ T-cells within the duodenal lamina propria.

IgA plays an important role in the immune defense of mucosal sites, where it is secreted at concentrations far in excess of other immunoglobulin classes. In this context, IgA prevents colonization and invasion by microorganisms, neutralizes bacterial toxins and is involved in the elimination of antigen from the subepithelial lamina propria. The IgA molecule is of particular relevance in the immunological defense of the gastrointestinal tract, and dysfunction of mucosal immunity contributes to a range of idiopathic inflammatory bowel diseases that occur in man and other species. Multiple IgA subclasses have been identified in humans, primates and lagomorphs, whereas mice, cattle and dogs have only a single subclass. The two human subclasses (IgA₁ and IgA₂) are defined by a difference in the length of the hinge region between the CH₁ and CH₂ domains. The single IgA subclass identified in dogs has a hinge region with a predicted amino acid sequence similar to the IgA₁ subclass of humans.

### BRIEF SUMMARY OF THE INVENTION

The present inventors have shown that dogs possess multiple IgA allelic variants. These variants differ principally within the 5′ end of the second exon of the α heavy chain gene, an area corresponding to the hinge region of the molecule. The presence of an extended hinge region of IgA makes it more susceptible to cleavage by proteases. Therefore differences in hinge length and composition between variants have significant effects on the function of the IgA molecules they encode. These differences result in certain genotypes having an increased susceptibility to disease caused by pathogens that produce proteases that can cleave the hinge region.

Accordingly, the invention provides a method for determining susceptibility to an IgA-related disorder in an animal, the method comprising:

a) identifying the or each IgA allelic variant present in a sample from the animal; and

b) thereby determining whether the animal is susceptible to an IgA-related disorder.

The invention further provides:
a probe, primer or antibody which is capable of detecting an IgA allelic variant;
a kit for carrying out the method of the invention comprising means for detecting an IgA allelic variant;
a method of preparing customized food for an animal which is susceptible to an IgA-related disorder, the method comprising:

(a) determining whether the animal is susceptible to an IgA-related disorder by a method of the invention; and

(b) preparing food suitable for the animal;
a method of providing a customized animal food, comprising providing food suitable for an animal which is susceptible to an IgA-related disorder to the animal, the animal's owner or the person responsible for feeding the animal, wherein the animal has been genetically determined to be susceptible to an IgA-related disorder;
a method for identifying an agent for the treatment of an IgA-related disorder, the method comprising:

(a) contacting an IgA allelic variant polypeptide or a polynucleotide which encodes an IgA allelic variant with a test agent; and

(b) determining whether the agent is capable of binding to the polypeptide or modulating the activity or expression of the polypeptide or polynucleotide;
a method of treating an animal for an IgA-related disorder, the method comprising administering to the animal an effective amount of a therapeutic compound which prevents or treats the disorder, wherein the animal has been identified as being susceptible to an IgA-related disorder by a method of the invention;
a database comprising information relating to IgA allelic variants and optionally their association with IgA-related disorder(s);
a method for determining whether an animal is susceptible to an IgA-related disorder, the method comprising:

(a) inputting data of one or more IgA allelic variant(s) present in the animal to a computer system;

(b) comparing the data to a computer database, which database comprises information relating to IgA allelic variants and the IgA-related disorder susceptibility associated with the variants; and

(c) determining on the basis of the comparison whether the animal is susceptible to an IgA-related disorder;
a computer program comprising program code that, when executed on a computer system, instructs the computer system to perform a method according to the invention;
a computer system arranged to perform a method according to the invention comprising:

(a) means for receiving data of the one or more IgA allelic variant(s) present in the animal;

(b) a module for comparing the data with a database comprising information relating to IgA allelic variants and the IgA-related disorder susceptibility associated with the variants; and

(c) means for determining on the basis of said comparison whether the animal is susceptible to an IgA-related disorder;
a method of preparing customized food for an animal which is susceptible to an IgA-related disorder, the method comprising:

(a) determining whether the animal is susceptible to an IgA-related disorder by a method according to the invention;

(b) electronically generating a customized animal food formulation suitable for the animal;

(c) generating electronic manufacturing instructions to control the operation of food manufacturing apparatus in accordance with the customized animal food formulation; and

(d) manufacturing the customized animal food according to the electronic manufacturing instructions;

an isolated polynucleotide comprising:

(a) an IgA variant sequence that differs to SEQ ID NO: 1 at one or more polymorphic positions as defined herein;

(b) any one of SEQ ID NO:s 3, 5, 7 or 9;

(c) a sequence that is complementary or is degenerate as a result of the genetic code to a sequence as defined in (a) or (b); or

(d) a fragment of (a), (b) or (c) which differs to SEQ ID NO: 1 at one or more polymorphic positions as defined in claim 5 and which is at least 10 nucleotides in length; and

a polypeptide comprising:

(a) a sequence encoded by a polynucleotide of the invention;

(b) any one of SEQ ID NO:s 4, 6, 8 or 10; or

(c) a fragment of (a) or (b) which differs to SEQ ID NO: 2 at one or more polymorphic positions as defined in claim 5 and which is at least 10 amino acids in length.

### BRIEF DESCRIPTION OF THE SEQUENCES

SEQ ID NO: 1 shows the polynucleotide sequence of the canine IgA α-chain, starting from the beginning of exon 1. SEQ ID NO: 2 shows the corresponding polypeptide sequence.

SEQ ID NO:s 3 and 4 show the polynucleotide and polypeptide sequences of variant A of the canine IgA α-chain.

SEQ ID NO:s 5 and 6 show the polynucleotide and polypeptide sequences of variant B of the canine IgA α-chain.

SEQ ID NO:s 7 and 8 show the polynucleotide and polypeptide sequences of variant C of the canine IgA α-chain.

SEQ ID NO:s 9 and 10 show the polynucleotide and polypeptide sequences of variant D of the canine IgA α-chain.

SEQ ID NO:s 11 to 17 show primer and probe sequences.

SEQ ID NO: 18 shows the Genbank sequence L36871 (canine IgA α-chain polynucleotide sequence).

### BRIEF DESCRIPTION OF THE DRAWINGS

FIG. 1 shows real-time RT-PCR quantification of mRNA expression in duodenal biopsies. The graph on the left (A) demonstrates the traces produced using primer set 1 showing the bimodal distribution of the expression. Samples either contained a relatively ‘high’ () or ‘low’ (□) amounts of α-chain mRNA. The graph on the right shows the overlap of the traces when primer set 2 is used indicating that similar amounts of α-chain mRNA is present in all samples but the forward primer in set 1 does not detect a significant portion of the mRNA in some samples.

FIG. 2 shows the four sequenced variants and their relationship to the Genbank sequence L36871. The numbering starts from the first base of exon 1 of the Genbank sequence. A single nucleotide polymorphism (C or T) (A) exists between individuals with the same variant which does not alter the predicted amino acid sequence. A variable number of CT repeats (D) are present in the intron sequence prior to the splice site of the second exon. A single base polymorphism at base 547 (T or A) (E) causes loss of the splice acceptor resulting in either long hinge variants (F to H) or short hinge variants (G to H). The first exon ends at base 306 (B) and it corresponds to the first domain of the heavy chain. Four bases in the intron sequence of all dogs sequenced do not agree with that of the Genbank sequence (C).

FIG. 3 shows the mRNA sequences for the IgA variants together with the positions of the primer sets used in the real-time PCR. The positions are numbered from the first base of exon one of the Genbank sequence. The position of the forward primer in set one includes the 9 base addition of variant C and D and would therefore not efficiently prime off this template. The forward primer in set 2 can amplify fragments from all variants. The difference in length of the RNA can be seen, with variant B the shortest and D the longest.

FIG. 4 shows the predicted amino acid sequence for the IgA variants based on the sequenced mRNAs. Position A corresponds to the (A-T) polymorphism in the first exon which has no effect on the encoded amino acid. The amino acid differences are highlighted by bold text. Variant B has the shortest hinge (7 AA) and variant D the longest (13 AA).

FIG. 5 shows an alignment of mRNA encoding human IgA₁ and IgA₂. The mRNA sequences encoding the human IgA subclasses are aligned to demonstrate the position of the 39 bp deletion of IgA₂ which results in the shorter hinge of this subclass. In contrast to the hinge variants of dogs, this deletion does include the initial bases of the second exon but is 5 bases from the 5′ end.

FIG. 6 shows a computer system arranged to perform a method according to the invention which comprises: means 20 for receiving genetic data from the animal; a module 30 for comparing the data with a database 10 comprising information relating to IgA allelic variants; and means 40 for determining on the basis of said comparison the susceptibility of the animal to an IgA-related disorder.

FIG. 7 shows the fluorescence donor probe and fluorescence acceptor probe sequences. Uppercase text indicate those bases included in the oligonucleotide probes and lowercase text are the bases located between the probe sequences. Identical bases are indicated by hyphens (-) and absent bases by blank spaces.

FIG. 8 shows examples of melting curves from sequenced plasmids containing each of the allelic variants of the IGHA gene. Equimolar concentrations of each plasmid were combined to produce the heterozygous mixes. Melt peaks corresponding to each of the gene variants present in heterozygous gene mixes could be detected for all possible allele combinations.

### DETAILED DESCRIPTION OF THE INVENTION

The primary functional role of IgA antibodies is to protect epithelial surfaces from infectious agents. Therefore, the principal sites of IgA synthesis and secretion are the gut, the respiratory epithelium, the lactating breast and other exocrine glands such as the salivary and tear glands. IgA antibodies are selectively transported across epithelia into sites such as the lumen of the gut, where they neutralize toxins and viruses and block the entry of bacteria across the intestinal epithelium. IgA antibodies are also secreted in breast milk (colostrum) and transferred to the gut of newborn offspring to provide protection against disease.

The IgA molecule is composed of two light chains and two heavy (α) chains. The heavy chain is divided into one variable domain (V_{H}) and three constant domains (C_{H}¹, C_{H}² and C_{H}³). The heavy chain also comprises a flexible stretch of polypeptide chain known as the hinge region, which is located between the C_{H}¹ and C_{H}² domains. The flexibility of the hinge region is required to allow both arms of the antibody molecule to bind sites that are different distances apart, and is also required for interaction with antibody-binding proteins that mediate immune effector mechanisms.

The present inventors have identified four novel allelic variants (allotypes) of canine IgA. These variants differ in the sequence of the α heavy chain gene, and in particular, in the coding region of the hinge. These differences result in variation in hinge length between each allelic variant. This variation is in part due to the presence of a single nucleotide polymorphism in the splice acceptor for the second exon at position 547 in relation to SEQ ID NO: 1. The presence of deoxythreonine (T) at position 547 results in a short hinge variant, such as variants A and B. The presence of deoxyadenosine (A) at position 547 abolishes the splice acceptor site, and therefore results in long hinge variants such as variants C and D. Variation in hinge length is also caused by other sequence differences in the coding region of the hinge, as discussed herein.

The presence of an extended hinge region of IgA makes it more susceptible to cleavage by proteases. Various pathogens secrete proteases that can cleave the hinge region of Ig molecules. Therefore, the presence of a long hinge variant of IgA in an animal may cause susceptibility to disease caused by such pathogens, for example gastrointestinal, skin and respiratory disease.

Accordingly, the present invention provides a method for determining susceptibility to an IgA-related disorder in an animal, the method comprising:

a) identifying the or each IgA allelic variant present in a sample from the animal; and

b) thereby determining whether the animal is susceptible to an IgA-related disorder.

The IgA-related disorder may be any disease, condition or disorder that is associated with IgA deficiency or dysfunction. Such a disorder is typically an immune related disorder or a bacterial overgrowth disorder in any epithelium where IgA is secreted, such as in the gut, skin or respiratory epithelium. Therefore, the IgA-related disorder is typically a gastrointestinal, skin, respiratory, rheumatoid or periodontal disease. In particular, the disease may be diarrhea, small intestinal bacterial overgrowth, inflammatory bowel disease, perianal fistulas, atopic dermatitis, pyoderma, anal furunculosis, malasessia infestans or disseminated aspergillosis.

The animal tested is typically a mammal, preferably a non-human animal, such as a dog, cat, horse, pig, cattle or sheep. The animal may be a companion animal or pet. In a preferred embodiment, the animal tested is a dog. The dog tested may be of any breed, or may be a mixed or crossbred dog, or an outbred dog (mongrel). The dog may be of a breed which is prone to IgA deficiency, such as the German Shepherd Dog, beagle, cocker spaniel, Irish Wolfhound, rottweiler or shar pei. Alternatively the dog tested may be of a breed which is particularly susceptible to skin disease, such as the shar pei, west highland white terrier, Labrador retriever, German Shepherd Dog or golden retriever, or a breed which is known to be susceptible to periodontal disease, such as maltese terriers, shih tsu, Yorkshire terriers, poodles and other small breed dogs.

In one embodiment, the dog tested is of a breed that is susceptible to gastrointestinal disease, such as the boxer, standard poodle, Labrador retriever, Golden retriever or Irish Wolfhound. German Shepherd dogs (also known as Alsatians) are particularly susceptible to a number of gastrointestinal diseases. Therefore, in a preferred embodiment, the dog tested by a method of the invention is of the German Shepherd Dog (GSD) breed. In another aspect, the dog may be a crossbred or outbred dog which is the result of a combination of the German Shepherd Dog breed and one or more other breeds.

In one embodiment of the invention, identification of the IgA allelic variant comprises detecting one or more polymorphisms in the hinge region of the IgA allelic variant, or a polymorphism which is in linkage disequilibrium with such a polymorphism. Preferably, such a polymorphism is at any one of the following positions in relation to SEQ ID NO: 1:

position 179 [C/T];

position 370 [T/C];

position 371 [T/C];

position 372 [C/G];

position 375 [G/T];

positions 514 to 546 [number of CT repeats];

position 547 [T/A];

position 563 [A/T];

positions 563 to 571 [deletion];

positions 576 to 578 [addition];

position 582 [C/T];

position 583 [A/G];

position 584 [T/A];

position 592 [G/A]; or

position 606 [G/A];

The present inventors have identified an association between non-specific dietary sensitivity and the presence of one or more variant C alleles. Therefore, in another preferred embodiment, the animal is tested for the presence of one or more C variant alleles, wherein the presence of at least one variant C allele indicates susceptibility to an IgA-related disorder.

The animal is preferably tested before any symptoms of an IgA-related disorder may be detected. The test may therefore be used to detect susceptibility to disease in an animal, in order to allow prevent the development or onset of an IgA-related disorder. Such preventative action may be related to medical treatment, dietary intervention or any other means of preventing or treating an IgA-related disorder as discussed herein. However, the test may also be used to aid or confirm a diagnosis of an IgA-related disorder in an animal.

### Detection of Allelic Variants

The detection of allelic variants according to the invention may comprise contacting an IgA polynucleotide or protein of the animal with a specific binding agent for an IgA variant and determining whether the agent binds to the polynucleotide or protein, wherein binding of the agent indicates the presence of the IgA variant, and lack of binding of the agent indicates the absence of the IgA variant.

The method is generally carried out in vitro on a sample from the animal. The sample typically comprises a body fluid and/or cells of the individual and may, for example, be obtained using a swab, such as a mouth swab. The sample may be a blood, urine, saliva, skin, cheek cell or hair root sample. The sample is typically processed before the method is carried out, for example DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the allelic variant(s).

In the present invention, any one or more methods may comprise determining the presence or absence of one or more IgA variants in the animal. The IgA variant is typically detected by directly determining the presence of the polymorphic sequence in a polynucleotide or protein of the animal. Such a polynucleotide is typically genomic DNA, mRNA or cDNA. The allelic variant may be detected by any suitable method such as those mentioned below.

A specific binding agent is an agent that binds with preferential or high affinity to the protein or polypeptide having the allelic variant but does not bind or binds with only low affinity to other polypeptides or proteins. The specific binding agent may be a probe or primer. The probe may be a protein (such as an antibody) or an oligonucleotide. The probe may be labelled or may be capable of being labelled indirectly. The binding of the probe to the polynucleotide or protein may be used to immobilize either the probe or the polynucleotide or protein.

Generally in the method, determination of the binding of the agent to the IgA variant can be carried out by determining the binding of the agent to the polynucleotide or protein of the animal. However in one embodiment the agent is also able to bind the corresponding wild-type sequence, for example by binding the nucleotides or amino acids which flank the allelic variant position, although the manner of binding to the wild-type sequence will be detectably different to the binding of a polynucleotide or protein containing the allelic variant.

The method may be based on an oligonucleotide ligation assay in which two oligonucleotide probes are used. These probes bind to adjacent areas on the polynucleotide which contains the allelic variant, allowing after binding the two probes to be ligated together by an appropriate ligase enzyme. However the presence of single mismatch within one of the probes may disrupt binding and ligation. Thus ligated probes will only occur with a polynucleotide that contains the allelic variant, and therefore the detection of the ligated product may be used to determine the presence of the allelic variant.

In one embodiment the probe is used in a heteroduplex analysis based system. In such a system when the probe is bound to polynucleotide sequence containing the allelic variant it forms a heteroduplex at the site where the allelic variant occurs and hence does not form a double strand structure. Such a heteroduplex structure can be detected by the use of single or double strand specific enzyme. Typically the probe is an RNA probe, the heteroduplex region is cleaved using RNAase H and the allelic variant is detected by detecting the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment a PCR primer is used that primes a PCR reaction only if it binds a polynucleotide containing the allelic variant, for example a sequence- or allele-specific PCR system, and the presence of the allelic variant may be determined by the detecting the PCR product. Preferably the region of the primer which is complementary to the allelic variant is at or near the 3′ end of the primer. The presence of the allelic variant may be determined using a fluorescent dye and quenching agent-based PCR assay such as the Taqman PCR detection system. In a preferred embodiment, one or more of the probes and/or primers shown in Table 4 (for example, all of the probes and primers in Table 4) are used in a Taqman assay to detect an allelic variant.

The specific binding agent may be capable of specifically binding the amino acid sequence encoded by a variant sequence. For example, the agent may be an antibody or antibody fragment. The detection method may be based on an ELISA system. The method may be an RFLP based system. This can be used if the presence of the allelic variant in the polynucleotide creates or destroys a restriction site that is recognized by a restriction enzyme.

The presence of the allelic variant may be determined based on the change which the presence of the allelic variant makes to the mobility of the polynucleotide or protein during gel electrophoresis. In the case of a polynucleotide single-stranded conformation allelic variant (SSCP) or denaturing gradient gel electrophoresis (DDGE) analysis may be used. In another method of detecting the allelic variant a polynucleotide comprising the polymorphic region is sequenced across the region which contains the allelic variant to determine the presence of the allelic variant.

In another embodiment of the invention, the presence of the allelic variant is detected by means of fluorescence resonance energy transfer (FRET). In particular, the variant may be detected by means of a dual hybridization probe system. This method involves the use of two oligonucleotide probes that are located close to each other and that are complementary to an internal segment of a target polynucleotide of interest, where each of the two probes is labelled with a fluorophore. Any suitable fluorescent label or dye may be used as the fluorophore, such that the emission wavelength of the fluorophore on one probe (the donor) overlaps the excitation wavelength of the fluorophore on the second probe (the acceptor). A typical donor fluorophore is fluorescein (FAM), and typical acceptor fluorophores include Texas red, rhodamine, LC-640, LC-705 and cyanine 5 (Cy5). The probe that is labelled with a donor fluorophore is referred to herein as the fluorescence donor probe, and the probe that is labelled at with an acceptor fluorophore is referred to herein as the fluorescence acceptor probe.

In order for fluorescence resonance energy transfer to take place, the two fluorophores need to come into close proximity on hybridization of both probes to the target. When the donor fluorophore is excited with an appropriate wavelength of light, the emission spectrum energy is transferred to the fluorophore on the acceptor probe resulting in its fluorescence. Therefore, detection of this wavelength of light, during excitation at the wavelength appropriate for the donor fluorophore, indicates hybridization and close association of the fluorophores on the two probes. Therefore, according to one embodiment, each probe is labelled with a fluorophore at one end such that the probe located upstream (5′) is labelled at its 3′ end, and the probe located downstream (3′) is labelled at is 5′ end. The gap between the two probes when bound to the target sequence may be from 1 to 20 nucleotides, preferably from 1 to 17 nucleotides, more preferably from 1 to 10 nucleotides, such as a gap of 1, 2, 4, 6, 8 or 10 nucleotides.

In one embodiment, the first of the two probes is designed to bind to a conserved sequence adjacent to a region of sequence variation between the allelic variants. Such a conserved region may be entirely conserved between the allelic variants, such that it has 100% identity with all allelic variants in that region. Alternatively, the first probe may have one or more mismatches with the sequence of one or more variant sequences. This conserved region is typically adjacent to a region of sequence variation between the allelic variants. The second probe binds to this region of sequence variation. In a preferred embodiment, the region of sequence variation is the hinge region of an allelic variant. The probe may be of any suitable length as discussed herein.

Polymorphisms within the sequence of the gene targeted by the second probe can be detected by measuring the change in melting temperature caused by the resulting base mismatches. The extent of the change in the melting temperature will be dependent on the number and base types involved in the nucleotide polymorphisms. The second probe typically has a different melting temperature for each allelic variant sequence, in order to distinguish one variant from each of the others. The melting temperatures of the probe located over the area of base variability are typically lower than the melting temperature of the probe located in an area of sequence similarity, due to the presence of base mismatches.

In order to detect allelic variants, the first and second probes as described herein are combined with a sample which comprises a polynucleotide comprising the target sequence. The target polynucleotide sequence corresponds to a region comprising the sequences to which the first and second probes hybridize, as discussed above, and which varies between allelic variants. Typically the target polynucleotide consists of DNA that has been amplified, for example using the polymerase chain reaction (PCR), from DNA extracted from the sample taken from the animal to be tested. Once the first and second probes have contacted the sample comprising the target polynucleotide, the temperature is varied across the range of the melting temperatures of the first and second probes, and the fluorescence emitted is detected using any suitable means. Therefore, for example, as a sample containing the labelled oligonucleotide probes and target polynucleotide is heated, the melting of the donor probe results in a decrease in the fluorescence emitted by the acceptor probe. By detecting the fluorescence emissions at various temperatures, the melting temperature of the second probe can be determined, and hence the presence of the or each allelic variant present in the sample can be detected.

### Polynucleotides

The invention also provides a polynucleotide which comprises an IgA variant sequence. An IgA variant sequence typically differs from SEQ ID NO: 1 (Genbank sequence L36871) at one or more of the following polymorphic positions:

position 179 [C/T];

position 370 [T/C];

position 371 [T/C];

position 372 [C/G];

position 375 [G/T];

positions 514 to 546 [number of CT repeats];

position 547 [T/A];

position 563 [A/T];

positions 563 to 571 [deletion];

positions 576 to 578 [addition];

position 582 [C/T];

position 583 [A/G];

position 584 [T/A];

position 592 [G/A]; or

position 606 [G/A];

Preferably the allelic variant sequence is variant A, B, C or D as discussed herein. Accordingly, a polynucleotide of the invention preferably comprises the sequence of SEQ ID NO: 3, SEQ ID NO: 5, SEQ ID NO: 7 or SEQ ID NO: 9 or a fragment thereof. The polynucleotide is typically at least 10, 15, 20, 30, 50, 100, 200 or 500 bases long, such as at least or up to 1 kb, 10 kb, 100 kb, 1000 kb or more in length. The polynucleotide will typically comprise flanking nucleotides on one or both sides of (5′ or 3′ to) the allelic variant, for example at least 2, 5, 10, 15 or more flanking nucleotides in total or on each side. Typically, the polynucleotide will be at least 95%, preferably at least 99%, even more preferably at least 99.9% identical to the polynucleotide sequences of SEQ ID NO: 3, 5, 7 or 9. Such numbers of substitutions and/or insertions and/or deletions and/or percentage identity may be taken over the entire length of the polynucleotide or over 50, 30, 15, 10 or less flanking nucleotides in total or on each side.

The polynucleotide may be RNA or DNA, including genomic DNA, synthetic DNA or cDNA. The polynucleotide may be single or double stranded. The polynucleotide may comprise synthetic or modified nucleotides, such as methylphosphonate and phosphorothioate backbones or the addition of acridine or polylysine chains at the 3′ and/or 5′ ends of the molecule.

A polynucleotide of the invention may be used as a primer, for example for PCR, or a probe. A polynucleotide or polypeptide of the invention may carry a revealing label. Suitable labels include radioisotopes such as ³²P or ³⁵S, fluorescent labels, enzyme labels or other protein labels such as biotin.

The invention also provides expression vectors that comprise polynucleotides of the invention and are capable of expressing a polypeptide of the invention. Such vectors may also comprise appropriate initiators, promoters, enhancers and other elements, such as for example polyadenylation signals which may be necessary, and which are positioned in the correct orientation, in order to allow for protein expression. Thus the coding sequence in the vector is operably linked to such elements so that they provide for expression of the coding sequence (typically in a cell). The term “operably linked” refers to a juxtaposition wherein the components described are in a relationship permitting them to function in their intended manner.

The vector may be for example plasmid, virus or phage vector. Typically the vector has an origin of replication. The vector may comprise one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. Mammalian promoters, such as β-actin promoters, may be used. Tissue-specific promoters are especially preferred. Viral promoters may also be used, for example the Moloney murine leukemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR).

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

Polynucleotides of the invention may be used as a probe or primer which is capable of selectively binding to an IgA variant. Preferably the probe or primer is capable of selectively binding to the polynucleotide sequence of SEQ ID NO: 3, 5, 7 or 9. The invention thus provides a probe or primer for use in a method according to the invention, which probe or primer is capable of selectively detecting the presence of an IgA variant. Preferably the probe is isolated or recombinant nucleic acid. It may correspond to or be antisense to the polynucleotide sequence of SEQ ID NO: 3, 5, 7 or 9. The probe may be immobilized on an array, such as a polynucleotide array.

The primers, probes and other fragments as described herein will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of a full length polynucleotide sequence of the invention.

In one embodiment of the invention, the probe is one of a pair suitable for use in a fluorescence resonance energy transfer (FRET) dual hybridization probe system, as discussed herein. In a preferred embodiment of the invention, the fluorescence donor and acceptor probes comprise or consist of the probe sequences set out in Table 8.

### Homologues

Homologues of polynucleotide or protein sequences are referred to herein. Such homologues typically have at least 70% homology, preferably at least 80, 90%, 95%, 97% or 99% homology, for example over a region of at least 15, 20, 30, 100 more contiguous nucleotides or amino acids. The homology may be calculated on the basis of nucleotide or amino acid identity (sometimes referred to as “hard homology”).

For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence, typically differs by at least 1, 2, 5, 10, 20 or more mutations, which may be substitutions, deletions or insertions of nucleotide or amino acids. These mutations may be measured across any of the regions mentioned above in relation to calculating homology. In the case of proteins the substitutions are preferably conservative substitutions. These are defined according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Shorter polypeptide sequences are also within the scope of the invention. For example, a fragment of a polypeptide sequence of the invention is typically at least 10, 15, 20, 30, 40, 50, 60, 70, 80, 100, 150 or 200 amino acids in length. In particular, this aspect of the invention encompasses the situation where the polypeptide is a fragment of a variant canine IgA heavy α chain which differs in amino acid sequence to a corresponding fragment of the non-variant sequence (i.e. SEQ ID NO: 1). A fragment of the variant IgA may be a Fv, F(ab′) or F(ab′)₂ fragment. A variant IgA fragment of the invention typically comprises the hinge region of the heavy chain.

Polypeptides of the invention may be chemically modified, for example post-translationally modified. The polypeptides may be glycosylated or comprise modified amino acid residues. Such modified polypeptides fall within the scope of the term “polypeptide” of the invention.

The polypeptides, polynucleotides, vectors, cells or antibodies of the invention may be present in an isolated or substantially purified form. They may be mixed with carriers or diluents which will not interfere with their intended use and still be regarded as substantially isolated. They may also be in a substantially purified form, in which case they will generally comprise at least 90%, e.g. at least 95%, 98% or 99%, of the proteins, polynucleotides, cells or dry mass of the preparation.

It is understood that any of the above features that relate to polynucleotides and proteins may also be a feature of the other polypeptides and proteins mentioned herein, such as the polypeptides and proteins used in the screening and therapeutic aspects of the invention. In particular such features may be any of the lengths, modifications and vectors forms mentioned above.

### Detector Antibodies

The invention also provides detector antibodies that are specific for a polypeptide of the invention. A detector antibody is specific for one IgA variant, for example, variant A, B, C or D, but does not bind to any other IgA variant. The detector antibodies of the invention are for example useful in purification, isolation or screening methods involving immunoprecipitation techniques.

Antibodies may be raised against specific epitopes of the polypeptides of the invention. An antibody, or other compound, “specifically binds” to a polypeptide when it binds with preferential or high affinity to the protein for which it is specific but does substantially bind not bind or binds with only low affinity to other polypeptides. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanized antibodies.

Antibodies may be used in a method for detecting polypeptides of the invention in a biological sample (such as any such sample mentioned herein), which method comprises:

I providing an antibody of the invention;

II incubating a biological sample with said antibody under conditions which allow for the formation of an antibody-antigen complex; and

III determining whether antibody-antigen complex comprising said antibody is formed.

Antibodies of the invention can be produced by any suitable method. Means for preparing and characterizing antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”. The fragment may be any of the fragments mentioned herein (typically at least 10 or at least 15 amino acids long).

A method for producing a polyclonal antibody comprises immunizing a suitable host animal, for example an experimental animal, with the immunogen and isolating immunoglobulins from the animal's serum. The animal may therefore be inoculated with the immunogen, blood subsequently removed from the animal and the IgG fraction purified. A method for producing a monoclonal antibody comprises immortalizing cells which produce the desired antibody. Hybridoma cells may be produced by fusing spleen cells from an inoculated experimental animal with tumor cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunization of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

### Detection Kit

The invention also provides a kit that comprises means for determining the presence or absence of one or more IgA allelic variant(s) in an animal. In particular, such means may include a specific binding agent, probe, primer, pair or combination of primers, or antibody, including an antibody fragment, as defined herein which is capable of detecting or aiding detection of an IgA allelic variant. The primer or pair or combination of primers may be sequence specific primers which only cause PCR amplification of a polynucleotide sequence comprising the IgA variant to be detected, as discussed herein. The kit may also comprise a specific binding agent, probe, primer, pair or combination of primers, or antibody which is capable of detecting the absence of the allelic variant. The kit may further comprise buffers or aqueous solutions.

The kit may additionally comprise one or more other reagents or instruments which enable any of the embodiments of the method mentioned above to be carried out. Such reagents or instruments may include one or more of the following: a means to detect the binding of the agent to the allelic variant, a detectable label such as a fluorescent label, an enzyme able to act on a polynucleotide, typically a polymerase, restriction enzyme, ligase, RNAse H or an enzyme which can attach a label to a polynucleotide, suitable buffer(s) or aqueous solutions for enzyme reagents, PCR primers which bind to regions flanking the allelic variant as discussed herein, a positive and/or negative control, a gel electrophoresis apparatus, a means to isolate DNA from sample, a means to obtain a sample from the individual, such as swab or an instrument comprising a needle, or a support comprising wells on which detection reactions can be carried out. The kit may be, or include, an array such as a polynucleotide array comprising the specific binding agent, preferably a probe, of the invention. The kit typically includes a set of instructions for using the kit.

### Screening for Therapeutic Agents

The present invention also relates to the use of variant IgA as a screening target for identifying therapeutic agents for the treatment of IgA-related disorders. In one embodiment the invention provides a method for identifying an agent useful for the treatment of IgA-related disorders, which method comprises contacting a variant IgA polypeptide or a polynucleotide with a test agent and determining whether the agent is capable of binding to the polypeptide or modulating the activity or expression of the polypeptide or polynucleotide. Any suitable binding assay format can be used to determine whether the IgA variant binds the test agent, such as the formats discussed below.

The method may be carried out in vitro, either inside or outside a cell, or in vivo. In one embodiment the method is carried out on a cell, cell culture or cell extract that comprises a variant IgA protein or polynucleotide. The cell may be any suitable cell, and is typically a cell in which the product is naturally expressed. For example, the cell may be a mucosal epithelial cell such as an IgA⁺ plasma cell from the duodenal lamina propria. The method may also be carried out in vivo in an non-human animal which is transgenic for an IgA variant polynucleotide. The transgenic non-human animal is typically of a species commonly used in biomedical research and is preferably a laboratory strain. Suitable animals include rodents, particularly a mouse, rat, guinea pig, ferret, gerbil or hamster. Most preferably the animal is a mouse.

The term “modulate” includes any of the ways mentioned herein in which the agent is able to modulate activity of an IgA variant polypeptide or polynucleotide. This may be determined by contacting the polypeptide or polynucleotide with the test agent under conditions that permit activity of the polypeptide or polynucleotide, and determining whether the test agent is able to modulate the activity of the polypeptide or polynucleotide.

The activity which is measured may be any of the activities which are mentioned herein, and may be the measurement of a property of the IgA variant polypeptide or polynucleotide, or an effect on a cellular component, cell or animal in which the method is being carried out. The effect may be one that is associated with an IgA-related disorder, and may be a characteristic or symptom of an IgA-related disorder, such as any such characteristic or symptom mentioned herein.

In one embodiment the assay measures the effect of the test agent on the binding between the variant IgA polypeptide or polynucleotide and another agent, such as a protease. In particular, the assay may include proteases from pathogens that are known to cause IgA-related disorders in dogs. Suitable assays in order to measure the changes in such interactions include fluorescence imaging plate reader assays, and radioligand binding assays. In the case where the activity is transcription from a gene the method may comprise measuring the ability of the candidate substance to modulate transcription, for example in a reporter gene assay.

Suitable candidate agents which may be tested in the above screening methods include antibody agents, for example monoclonal and polyclonal antibodies, single chain antibodies, chimeric antibodies and CDR-grafted antibodies. Furthermore, combinatorial libraries, defined chemical identities, peptide and peptide mimetics, oligonucleotides and natural agent libraries, such as display libraries may also be tested. The test agents may be chemical compounds, which are typically derived from synthesis around small molecules which may have any of the properties of the agent mentioned herein. Batches of the candidate agents may be used in an initial screen of, for example, ten substances per reaction, and the substances of batches which show modulation tested individually. The term ‘agent’ is intended to include a single substance and a combination of two, three or more substances. For example, the term agent may refer to a single peptide, a mixture of two or more peptides or a mixture of a peptide and a defined chemical entity.

In one aspect of the invention, the test agent is a food ingredient. Hence, the invention relates to a method of screening food ingredients to determine whether they contribute to or aggravate gastrointestinal disease in susceptible animals, or if they prevent or alleviate gastrointestinal disease. The food ingredient may be one that is typically used in animal or pet food or other types of food, or may be a novel food ingredient.

The present invention also provides an agent identified by a screening method of the invention. An agent identified in the screening method of the invention may be used in the therapeutic treatment of an IgA-related disorder. Such an agent may be formulated and administered in any means or amounts as discussed below.

### Treatment of IgA-Related Disorders

The invention provides a method of treating an animal for an IgA-related disorder, the method comprising identifying an animal which is susceptible to an IgA-related disorder by a method of the invention, and administering to the animal an effective amount of a therapeutic agent which treats the IgA-related disorder. The IgA-related disorder may be any disease or disorder mentioned herein, and is typically a gastrointestinal, skin, respiratory disease, rheumatoid or periodontal disease. The therapeutic agent is typically a drug such as an anti-inflammatory (e.g. sulphur salasine), a corticosteroid (e.g. prednisolone), an antibiotic (e.g. amoxycillin or enrofloxacin) or a protease inhibitor (e.g. amprenavir). The therapeutic agent may be any drug known in the art that may be used to treat an IgA-related disorder, or may an agent identified by a screening method as discussed previously.

The therapeutic treatment may result in a change of the bacterial flora of the animal. In particular, the bacterial flora of the animal may be altered to reduce the production of proteases that can degrade IgA. Such a change is typically effected in the gastrointestinal system of the animal, and may be carried out by administering agents such as prebiotics or probiotics to the animal.

The therapeutic agent may be administered in various manners such as orally, intracranially, intravenously, intramuscularly, intraperitoneally, intranasally, intrademally, and subcutaneously. The pharmaceutical compositions that contain the therapeutic agent will normally be formulated with an appropriate pharmaceutically acceptable carrier or diluent depending upon the particular mode of administration being used. For instance, parenteral formulations are usually injectable fluids that use pharmaceutically and physiologically acceptable fluids such as physiological saline, balanced salt solutions, or the like as a vehicle. Oral formulations, on the other hand, may be solids, for example tablets or capsules, or liquid solutions or suspensions. In a preferred embodiment, the therapeutic agent is administered to the animal in its diet, for example in its drinking water or food.

The amount of therapeutic agent that is given to an animal will depend upon a variety of factors including the condition being treated, the nature of the animal under treatment and the severity of the condition under treatment. A typical daily dose is from about 0.1 to 50 mg per kg, preferably from about 0.1 mg/kg to 10 mg/kg of body weight, according to the activity of the specific inhibitor, the age, weight and conditions of the animal to be treated, the type and severity of the disease and the frequency and route of administration. Preferably, daily dosage levels are from 5 mg to 2 g.

### Customized Food

In one aspect, the invention relates to a customized diet for an animal that is susceptible to an IgA-related disorder. In a preferred embodiment, the customized food is for a companion animal or pet, such as a dog. Such a food may be in the form of, for example, wet pet foods, semi-moist pet foods, dry pet foods and pet treats. Wet pet food generally has a moisture content above 65%. Semi-moist pet food typically has a moisture content between 20-65% and can include humectants and other ingredients to prevent microbial growth. Dry pet food, also called kibble, generally has a moisture content below 20% and its processing typically includes extruding, drying and/or baking in heat. The ingredients of a dry pet food generally include cereal, grains, meats, poultry, fats, vitamins and minerals. The ingredients are typically mixed and put through an extruder/cooker. The product is then typically shaped and dried, and after drying, flavors and fats may be coated or sprayed onto the dry product.

Accordingly, the present invention enables the preparation of customized food suitable for an animal which is susceptible to an IgA-related disorder, wherein the customized animal food formulation comprises ingredients that prevent or alleviate IgA-related disorders, and/or does not comprise components that contribute to or aggravate IgA-related disorders. Such ingredients may be any of those known in the art to prevent or alleviate an IgA-related disorder. Alternatively, screening methods as discussed herein may identify such ingredients. The preparation of customized animal food may be carried out by electronic means, for example by using a computer system.

In one embodiment, the customized food may be formulated to alter the profile of food proteins in order to minimize the potential for secondary dietary sensitivity. The customized food may be hypoallergenic or may exclude ingredients that are poorly tolerated or cause allergies, for example gluten-containing grains such as wheat, particular protein sources such as animal proteins, milk (lactose), eggs, soy, peanuts, shellfish, fruits or tree nuts.

In another embodiment, the customized food may be formulated to include functional or active ingredients that help prevent or alleviate an IgA-related disorder. Such an ingredient may be a compound that stimulates immune function or protects against degradation of IgA, for example β-glucans or glutamine. Alternatively, exogenous IgA may be administered orally in the diet, for example using colostrum or eggs. These may be hyperimmunized to pathogens, such as pathogenic E. coli (e.g. EPEC), Campylobacter or Salmonella.

The functional or active ingredient may help to prevent or alleviate an IgA-related disorder by improving gut barrier function, for example the functional ingredient may be a prebiotic, probiotic or oligosaccharide. In one aspect, the customized food is formulated to prevent or alleviate IgA-related skin disease. Such food may comprise functional ingredients that improve the condition of the skin, for example vitamin C, taurine, curcumin or aloe vera. One particular example of a skin support diet is described in International Patent Application No. PCT/GB02/02538. Other active ingredients that may be added to a customized food include lutein, lycopene, β-carotene, eicosapentaenoic acid (EPA) and docosahexaenoic acid (DHA).

The present invention also relates to a method of providing a customized animal food, comprising providing food suitable for an animal which is susceptible to an IgA-related disorder to the animal, the animal's owner or the person responsible for feeding the animal, wherein the animal has been determined to be susceptible to an IgA-related disorder by a method of the invention. In one aspect of the invention, the customized food is made to inventory and supplied from inventory, i.e. the customized food is pre-manufactured rather than being made to order. Therefore according this aspect of the invention the customized food is not specifically designed for one particular animal but instead is suitable for more than one animal. For example, the customized food may be suitable for any animal that is susceptible to an IgA-related disorder. Alternatively, the customized food may be suitable for a sub-group of animals that are susceptible to an IgA-related disorder, such as animals of a particular breed, size or lifestage. In another embodiment, the food may be customized to meet the nutritional requirements of an individual animal.

In a preferred aspect of the invention, the customized food is suitable for a dog. In one embodiment, the customized food comprises a base diet and an immune support gravy which is specifically designed for an animal that is susceptible to an IgA-related disorder. The gravy may comprise active ingredients such as curcumin, aloe vera, taurine, vitamin C, lutein, lycopene, β-carotene, EPA, DHA or any other functional ingredient as discussed herein.

In another embodiment, the customized food consists of a primary kibble and secondary kibble. The primary kibble typically contains non-irritant ingredients, for example proteins derived from fish and carbohydrates derived from rice, in order to minimize dietary sensitivity. The primary kibble may therefore comprise ingredients such as protein, fat, linoleic and arichodonic acids, minerals and vitamins. The secondary kibble typically comprises active or functional ingredients that help to prevent an immune response arising to the food. The secondary kibble may therefore comprise ingredients such as aloe vera, curcumin, taurine, EPA, DHA, vitamin C or any other active or functional ingredient as discussed herein. The customized food will typically be a mixture of the primary and secondary kibbles in which the primary kibble is present in a larger quantity than the secondary kibble. For example, the customized food may consist of from 60% to 99% of primary kibble, such as at least 70%, 80%, 85%, 90% or 95% primary kibble, where the remainder of the customized food is the secondary kibble. Preferably, the customized food consists of about 90% primary kibble and about 10% secondary kibble.

### Bioinformatics

The sequences of the IgA variants may be stored in an electronic format, for example in a computer database. Accordingly, the invention provides a database comprising information relating to IgA allelic variant sequences. The database may include further information about the allelic variant, for example the level of association of the allelic variant with an IgA-related disorder or the frequency of the allelic variant in the population. In one aspect of the invention, the database further comprises information regarding the food components which are suitable and the food components which are not suitable for animals who possess a particular allelic variant of IgA.

A database as described herein may be used to determine the susceptibility of an animal to an IgA-related disorder. Such a determination may be carried out by electronic means, for example by using a computer system (such as a PC). Typically, the determination will be carried out by inputting genetic data from the animal to a computer system; comparing the genetic data to a database comprising information relating to IgA allelic variants; and on the basis of this comparison, determining the susceptibility of the animal to an IgA-related disorder.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

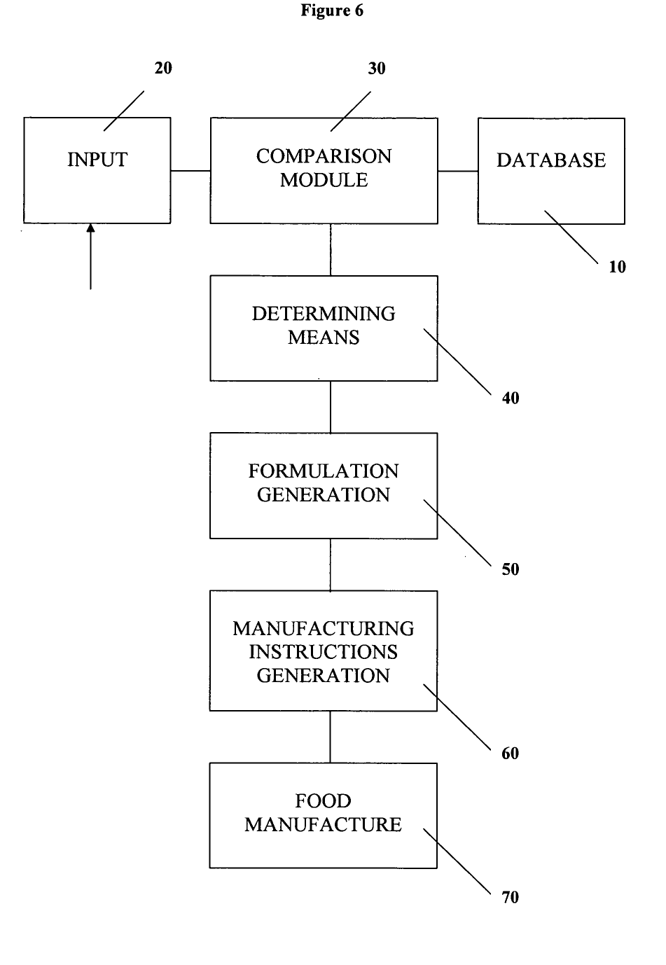

As illustrated in FIG. 6, the invention also provides an apparatus arranged to perform a method according to the invention. The apparatus typically comprises a computer system, such as a PC. In one embodiment, the computer system comprises: means 20 for receiving genetic data from the animal; a module 30 for comparing the data with a database 10 comprising information relating to IgA allelic variants; and means 40 for determining on the basis of said comparison the susceptibility of the animal to an IgA-related disorder.

### Food Manufacturing

In one embodiment of the invention, the manufacture of a customized animal food may be controlled electronically. Typically, information relating to the IgA allelic variant(s) present in an animal may be processed electronically to generate a customized animal food formulation. The customized animal food formulation may then be used to generate electronic manufacturing instructions to control the operation of food manufacturing apparatus. The apparatus used to carry out these steps will typically comprise a computer system, such as a PC, which comprises means 50 for processing the nutritional information to generate a customized animal food formulation; means 60 for generating electronic manufacturing instructions to control the operation of food manufacturing apparatus; and a food product manufacturing apparatus 70.

The food product manufacturing apparatus used in the present invention typically comprises one or more of the following components: container for dry pet food ingredients; container for liquids; mixer; former and/or extruder; cut-off device; cooking means (e.g. oven); cooler; packaging means; and labelling means. A dry ingredient container typically has an opening at the bottom. This opening may be covered by a volume-regulating element, such as a rotary lock. The volume-regulating element may be opened and closed according to the electronic manufacturing instructions to regulate the addition of dry ingredients to the pet food.

Dry ingredients typically used in the manufacture of pet food include corn, wheat, meat and/or poultry meal. Liquid ingredients typically used in the manufacture of pet food include fat, tallow and water. A liquid container may contain a pump that can be controlled, for example by the electronic manufacturing instructions, to add a measured amount of liquid to the pet food.

In one embodiment, the dry ingredient container(s) and the liquid container(s) are coupled to a mixer and deliver the specified amounts of dry ingredients and liquids to the mixer. The mixer may be controlled by the electronic manufacturing instructions. For example, the duration or speed of mixing may be controlled. The mixed ingredients are typically then delivered to a former or extruder. The former/extruder may be any former or extruder known in the art that can be used to shape the mixed ingredients into the required shape. Typically, the mixed ingredients are forced through a restricted opening under pressure to form a continuous strand. As the strand is extruded, it may be cut into pieces (kibbles) by a cut-off device, such as a knife. The kibbles are typically cooked, for example in an oven. The cooking time and temperature may be controlled by the electronic manufacturing instructions. The cooking time may be altered in order to produce the desired moisture content for the food. The cooked kibbles may then be transferred to a cooler, for example a chamber containing one or more fans.

The food manufacturing apparatus may comprise a packaging apparatus. The packaging apparatus typically packages the food into a container such as a plastic or paper bag or box. The apparatus may also comprise means for labelling the food, typically after the food has been packaged. The label may provide information such as: ingredient list; nutritional information; date of manufacture; best before date; weight; and species and/or breed(s) for which the food is suitable.

The invention is illustrated by the following Examples:

### EXAMPLE 1

### Materials and Methods

### Sample Collection

Endoscopic biopsies of duodenal mucosa were obtained from dogs presented to the Department of Clinical Veterinary Science, University of Bristol for investigation of gastrointestinal disease. Gastroduodenoscopy was performed using a GIF-XQ230 flexible video endoscope (Olympus Keymed, Southend-on-Sea, UK). Multiple mucosal biopsies were taken at the level of the caudal duodenal flexure using FB-25K biopsy forceps (Olympus Keymed). Biopsies were placed in a 1.0-ml cryotube (NUNC, Fischer Scientific Ltd., Loughborough, Leicestershire), snap frozen in liquid nitrogen and stored at −70° C.

### RNA/DNA Isolation

Two endoscopic biopsies (total tissue mass 9-16 mg) were added to a green Ribolyser tube (Ribolyser System, Thermo-Hybaid, Ashford, Middlesex, UK) containing 400 μl of lysis buffer from the RNA isolation kit (see below) and processed for 45 seconds at 6.0 m/s to homogenize the biopsies. This lysate was processed through the RNeasy Isolation System (Qiagen Ltd., Crawley, U.K.) as per the manufacturer's protocol except that the RNA was eluted in 100 μl of nuclease-free waster. This procedure produces a mixture of total RNA and a significant amount of genomic DNA. A negative control of nuclease free water was passed through the extraction procedure. Samples were stored at −70° C. prior to use.

### DNase Digestion

DNase digestion was carried out on 50 μl of the RNA/DNA mix from the extraction detailed above. DNase digestion was carried out both in solution and on the subsequent Qiagen column used to further purify the RNA. In-solution DNase digestion was carried out by treating 50 μl of the extraction mix with 5 units of Amplification Grade DNase 1 (Invitrogen Ltd.) as per the manufacturer's instructions. In order to remove any residual DNase or EDTA from the treated RNA, the solution was passed through the RNeasy Isolation System (Qiagen Ltd.) a second time using the RNA clean-up protocol. A second DNase digestion was carried out on this column using the RNase-Free DNase Set (Qiagen Ltd.).

### Primer Design

Primers for production of the cloned RT-PCR and PCR products were designed using Primer 3 (Rozen S, Skaletzky H J (2000) Primer3 on the WWW for general users and for biologist programmers. Humana Press, Totowa, N.J.) against the Genbank sequence for canine α-chain (accession number: L36871) and are shown in Table 1. The forward primer was located in the 3′ end of exon 1 and the reverse primer in the 5′ end of exon 2 which together produced a 550 bp DNA product and a 300 bp product with cDNA. The primer and probe sequences for identification of suitable dogs to clone DNA and RNA sequences for α-chain from have been used previously for the quantification of α-chain transcript from canine endoscopic duodenal mucosal biopsies and are shown in Table 1 (α-chain set 1).

This original set of primers was designed such that the forward primer spanned the junction between the first and second exon. A second set of primers was designed using Primer3 with the same probe sequence, such that the forward primer was located in the first exon (α-chain set 2). The primer and probe set was designed such that the annealing temperatures of the primers were 60° C. and the probe 8-10° C. higher, and that a product of between 80 and 200 bases in length would be obtained. In order to minimize primer-dimer formation, the maximum self-complementarity was 6 and the maximum 3′ self-complementarity was 2.

TABLE 1

Primer and Probe Sequences

| | | Forward | Reverse | | | |
|---|---|---|---|---|---|---|
| | Product | Primer | Primer | 5′- | Probe Sequence | 3′- |
| Primer Set | Length | (5′-3′) | (5′-3′) | Fluorophore | (5′-3′) | Quencher |
| Cloned | 550 bp gDNA | TGTGAAC | GCATTGG | Texas Red | GTCATCCATG | BHQ-2 |
| Product | 300 bp cDNA | GTGACCT | AGCCTAA | | TCCCTCGTGC | |
| Primers | | GGAATG | AAGCAG | | AATGAG | |
| α-Chain | 84 bp cDNA | TGTGCCCT | AGGGCTG | | | |
| Set 1 | | GCAAAGA | GCTTCTGT | | | |
| | | TAACA | AGTGA | | | |
| α-Chain | 136 bp cDNA | CGTCTGTG | AGGGCTG | | | |
| Set 2 | | AAATGCC | GCTTCTGT | | | |
| | | AAGTG | AGTGA | | | |

### RT-PCR

Gene specific real-time RT-PCR amplification of α-chain was performed using the Platinum Quantitative RT-PCR Thermoscript™ One-Step System (Invitrogen Ltd) using 5 μl of RNA, 4.5 mM Mg²⁺ and the primers and probe at a concentration of 200 nM and 100 nM respectively, in a final volume of 25 μl. No-RT reactions were made by substituting the Thermoscript enzyme mix with 2 units of Platinum Taq DNA Polymerase (Invitrogen Ltd.). The negative control from the extraction procedure, as well as a nuclease-free water control, were included with all sample runs. The RT-PCR was performed in an iCycler IQ (Bio-Rad Laboratories Ltd, Hercules, Calif.) with an initial incubation of 55° C. for 20 minutes (α-chain), followed by 95° C. for 5 minutes and then 45 cycles of 95° C. for 10 seconds, 60° C. for 15 seconds during which the fluorescence data were collected. All reactions were made up on ice and placed in the iCycler held at the initial incubation temperature to minimize primer-dimer formation. The threshold cycle (Ct value) was calculated as the cycle when the fluorescence of the sample exceeded a threshold level corresponding to 10 standard deviations from the mean of the baseline fluorescence.

RT-PCR to produce the products for cloning was performed using the Platinum Quantitative RT-PCR Thermoscript™ One-Step System (Invitrogen Ltd) using 5 μl of RNA as described previously. DNA amplification was performed by substituting the Thermoscript enzyme mix with 2 units of Platinum Taq DNA Polymerase (Invitrogen Ltd.) with 5 μl of the RNA/DNA mix. The PCR protocol was altered for the longer products so that after the initial incubation, 45 cycles of 95° C. for 15 seconds, 60° C. for 30 seconds and 72° C. for 30 seconds were performed followed by a final incubation of 72° C. for 5 minutes. These products were separated using 2% agarose gel electrophoresis and appropriately sized bands were excised from the gel and then purified using the QIAquick PCR Purification Kit (Qiagen Ltd., Crawley, UK) following the manufacturer's instructions.

### Cloning and Sequencing of Products

The purified PCR products were cloned using the TOPO TA Cloning kit (Invotrogen Ltd.) as per the manufacturer's instructions using chemically competent E. Coli. Following white/blue colony selection, positive clones were sub-cultured overnight and the plasmids were purified using the Qiagen Plasmid Mini Prep Kit (Qiagen Ltd.) as per the manufacturer's instructions. At least four clones per reaction mix (e.g. DNA and RNA) were purified for each dog and were sent to the Sequencing Service (School of Life Sciences, University of Dundee, Dundee, Scotland) for sequencing. Sequence results were aligned with the Genbank sequence using Omiga 2.0 (Accelrys, Cambridge, UK).

### Results

### IgA Expression

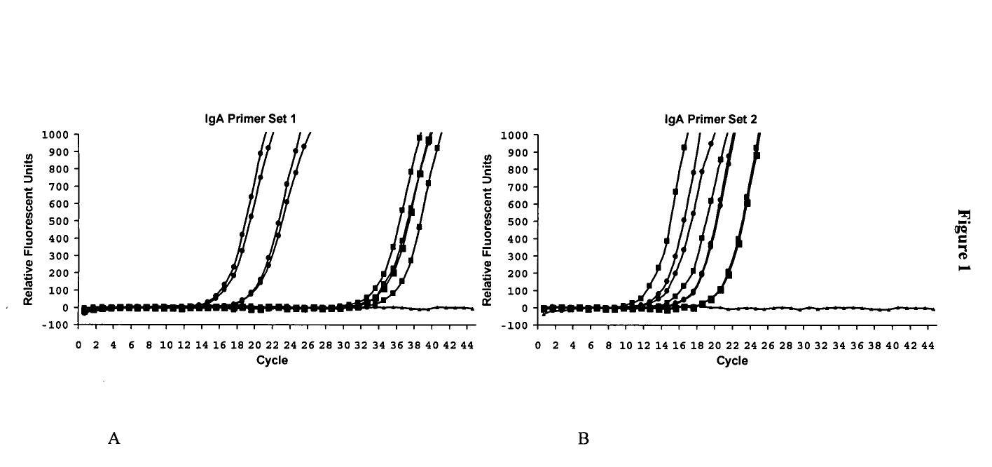

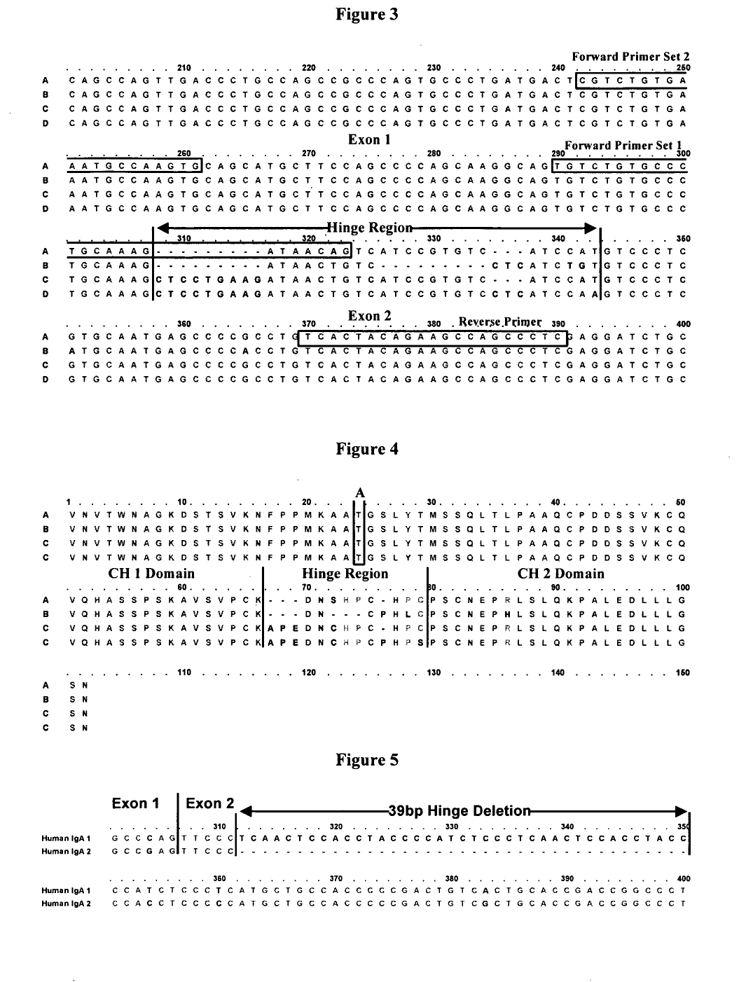

A bimodal pattern of expression of IgA mRNA from canine duodenal mucosa was previously shown (FIG. 1A) using primer set 1 (Peters, I. R., C. R. Helps, R. M. Batt, M. J. Day, and E. J. Hall. 2003. Quantitative real-time RT-PCR measurement of mRNA encoding alpha-chain, pIgR and J-chain from canine duodenal mucosa. J Immunol Methods 275:213). To confirm the quantification data, a second set of primers were designed (Table 1). Surprisingly, when primer set 2 was used, the bimodal distribution was lost (FIG. 1B). The two primer sets differ only in the position of the forward primer, which spans the junction between exon one and two, as shown in FIG. 3. These results indicated that a similar amount of α-chain mRNA is present in all samples but that the forward primer in set 1 does not detect a significant portion of the mRNA in some samples. Therefore, a primer set was designed which amplified a segment of both gDNA and cDNA which encompassed this region, in order to determine whether there were any sequence differences.

### Sequencing Data

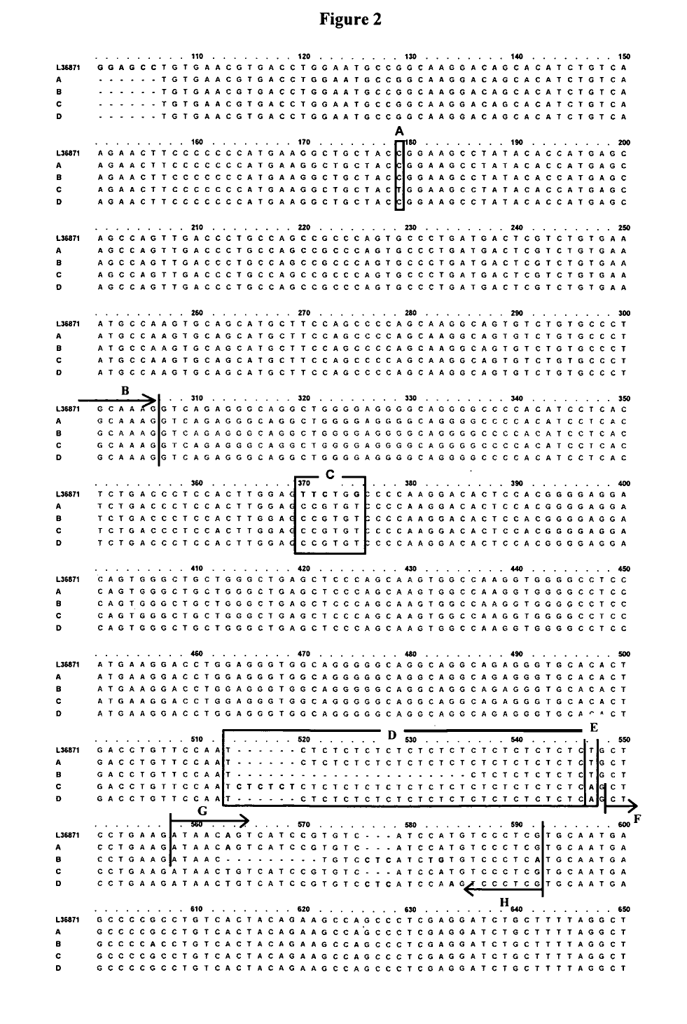

Four alternate sequences for the 5′-end of the second exon of the dog alpha heavy chain gene were identified, three of which have not previously been described (FIG. 2). The variants were termed A to D, with variant A being similar to the Genbank sequence and thus the variant that was detected using primer set 1. A single nucleotide polymorphism exists at position 179 which differs between individuals with the same variant but this polymorphism does not alter the encoded amino acid (FIG. 4).

A major difference between the variants was the position of the splice acceptor site for the second exon. This difference in the splice acceptor site was due to a single base polymorphism at position 547, that resulted in loss of a splice acceptor site for the second exon (FIG. 2). The presence of deoxyadenosine at this point resulted in coding for the mRNA from position 549 (variants C and D), whereas the presence of a deoxythreonine at this position led to coding from position 558 (variants A and B) with a transcript that is nine bases shorter.

The shortest variant was variant B which had the deoxythreonine polymorphism at position 558 but also had a nine base deletion within the 5′ end of the second exon (positions 563 to 571) and three additional bases after this deletion (positions 576 to 578). This variant also had other base differences at positions 582, 583, 592 and 606 compared with the other variants, and this resulted in alteration in the predicted amino acid sequence. The major difference between variants C and D was the presence of a three base addition similar to that in variant B between positions 576 to 578, making variant D the longest of the four.

The combinations of variants found in each individual dog are detailed in Table 2. These results indicate that more than one variant can be detected in some individual dogs, suggesting that heterozygous individuals exist. There is also evidence that both variants possessed by an individual are transcribed as the sequences were detected in both gDNA and cDNA products with the exception of * and +. These were only found in the gDNA (*) and cDNA (+) products respectively. All variants were found in two or more individuals with the exception of variant D which was only found in dog 48.

TABLE 2
Variant of IgA found in each dog sequenced

| Dog | Breed | Variant A | Variant B | Variant C | Variant D |
|---|---|---|---|---|---|
| 34 | Labrador | — | X | X (*) | — |
| 40 | GSD | — | — | X | — |
| 41 | GSD | — | — | X | — |
| 43 | Staffordshire Bull | X | X | — | — |
| | Terrier | | | | |
| 45 | Greyhound | X | — | X (+) | — |
| 46 | Crossbred | X | X | — | — |
| 47 | Border Collie | — | — | — | X |
| 48 | Cocker Spaniel | — | — | X | |

### EXAMPLE 2

### Materials and Methods

### DNA Samples

Buccal cells were collected from dogs by rotating a sterile cytology brush (Rocket Medical, Cat No. R57483) six times in the inside of the cheek. The brushes were then replaced in their individual wrapper and left to dry for a minimum of two hours at room temperature. DNA was extracted using the Qiagen QIAamp DNA Blood Mini Kit (Cat No. 51104) following the Buccal Swab Spin protocol. The DNA was eluted using 100 μl of dH₂O and then stored at −20° C.

### PCR Amplification

Primers were designed using Primer 3 (Rozen S, Skaletzky H J (2000) Primer3 on the WWW for general users and for biologist programmers. Humana Press, Totowa, N.J.) against the Genbank sequence for the α-chain (accession number: L36871). The forward primer is located in the 3′ end of exon 1 and the reverse primer in the 5′ end of exon 2 which together produced a 550 bp DNA product. The oligos were ordered from Sigma-Genosys, desalted, and used at 0.025 μM synthesis scale. 50 μl PCR reactions were carried using 25 pmol of each oligo and 50 ng of DNA from each dog sample and 25 μl of Eurogentec HotGoldstar PCR mastermix containing a red loading dye and 1.5 mM MgCl (PK-0073-02R).

Thermal cycling was performed using a Hybrid MBS 0.2S PCR machine using the following cycling conditions: incubation at 95° C. for 10 min, followed by 10 cycles of 95° C. for 30 sec, 64° C. (−1° C. per cycle) for 45 sec and 72° C. for 90 sec, followed by 28 cycles of 95° C. for 30 sec, 55° C. for 45 sec and 72° C. for 90 sec. 5 μl of each of the PCR sample and 1 μl of Gelstar nucleic acid gel stain (BioWhittaker Molecular Applications, Cat. No 50535) were run on a 2% agarose (Invitrogen, Cat. No 15510-027) gel at 100 mV to check for product. Successful PCR products were purified using a 96 well PCR cleanup plate (Millipore, Cat No MANU03010) following the standard method. The extra wash phase with 50 μl of H₂0 was added to remove red dye from the mastermix. Samples were quantified using 1 μl of purified PCR product on the Nanodrop Spectrophotometer. Analysis carried out using nucleic acid sample DNA-50 on the Nanodrop 2.4.7a software.

### DNA Sequencing

Cycle sequencing was performed using 25 fmol of purified PCR product with the CEQ 2000 Dye Terminator Cycle Sequencing with Quick Start kit (Beckman Coulter, P/N 608120). 20 μl reactions were carried out using 3 μl of DTCS quick Start Master Mix, 1 μl of forward or reverse primer that was used in the PCR step (5 pmol) and an adjustable volume of DNA template and dH₂0. Thermal cycling was performed using the same PCR machine as used in the PCR step but under the following conditions: 30 cycles of 96° C. for 20 sec, 50° C. for 20 sec and 60° C. for 4 min. Following these cycles, the samples were subjected to ethanol precipitation, and were evaporated for dryness using a vacuum pump for 40 min. Samples were resuspended in 40 μl of deionized formamide and a drop of mineral oil was placed on top. The samples were run on a Beckman CEQ 2000 Sequencer using the LFR capillary method. The sequence traces were analyzed using the CEQ200XL DNA Analysis System software Version 4.3.9.

### Results

In order to investigate the distribution of IgA alleles within dog breeds, the IgA genotype of 183 dogs from 11 different breeds was determined. The IgA genotypes for each dog breed are shown in Table 3 below. All 54 German Shepherd dogs tested were homozygous for variant C.

TABLE 3
Breed study results using sequencing method

| | Homozygous | | | | Heterozygous | | | | | |
|---|---|---|---|---|---|---|---|---|---|---|
| Breed | AA | BB | CC | DD | AB | AC | AD | BC | BD | CD |
| German Shepherd | | | 54 | | | | | | | |
| Labrador retriever | | 8 | 1 | | 1 | | 1 | 5 | | |
| Shih Tzu | 12 | 1 | 2 | | | | | | | |
| Rottweiler | 15 | | | | | | | | | |
| Golden retriever | | 7 | 4 | | | | | 4 | | 1 |
| Beagle | 1 | 1 | 2 | 6 | | | | | | 1 |
| Dobermann | 5 | | 10 | | | | | | | |
| Yorkshire Terrier | 6 | 1 | 4 | | | 1 | | 1 | | 1 |
| King Charles | | 5 | | | 5 | | | | | |
| Cavalier Spaniel | | | | | | | | | | |
| West Highland | 8 | | 3 | | 1 | | | | | |
| White Terrier | | | | | | | | | | |
| American Cocker | 1 | | 4 | | | | | | | |
| Spaniel | | | | | | | | | | |

### EXAMPLE 3

### Materials and Methods

### Template

The allelic identities of canine DNA samples were determined using the 5′-3′ exonuclease (“TaqMan”) assay. Canine genomic DNA was used as the template. The DNA was used either as a direct isolate, or in the case of very dilute samples, was amplified using GenomiPhi™ DNA Amplification Kit (Amersham Biosciences). Assays were performed using DNA at a concentration of 50-100 ng/μl.

### Reaction

Reactions were carried out using: 12.5 μl TaqMan Universal PCR Master Mix, no AmpErase UNG (Applied Biosystems, Warrington, UK); 1 μl each oligonucleotide (10 pm/μl stock solution) (Sigma Genosys, Cambs, UK); 1 μl TaqMan probe (5 pm/μl stock solution) (Sigma Genosys, Cambs, UK); 9.5 μl nuclease free water; and 1 μl template DNA. The probes were labelled 5′ with 6FAM and 3′ with TAMRA. Reactions were performed in an ABI 7700 Sequence Detection System using default PCR conditions, with the exception of the annealing temperature, which was raised to 63° C. Primers and probe sequences are shown in Table 4.

TABLE 4

Primer and Probe sequences

| | | | |
|---|---|---|---|
| Allele | Forward primer | Reverse primer | Probe |

| | | | |
|---|---|---|---|
| A | GAGGGTGCACACTGA | CACGAGGGACATGGA | CTCTCTCTGCTCCTGAAGA |
| | CCTGTT | TGAC | TAACAGTCATCCGT |
| B | GCACACTGACCTGTTC | GGGCTGGCTTCTGTAG | ATAACTGTCCTCATCTGTG |
| | CAATCTC | TGACA* | TCCCTCATGCA |
| C | GAGGGTGCACACTGA | CACGAGGGACATGGA | CTCTCTCAGCTCCTGAAGA |
| | CCTGTT | TGAC* | TAACTGTCATCCGT |
| D | TCTCTCTCAGCTCCTG | GGGCTGGCTTCTGTAG | CCGTGTCCTCATCCAAGTC |
| | AAGATAACTG | TGACA | CCTCG |

*The table shows corrected sequences for two of the reverse primers. The incorrect sequences previously given were: GCACACTGACCTGTTCCAATCTC (allele B reverse primer); and GGGCTGGCTTCTGTAGTGACA (allele C reverse primer).

### Results

The IgA genotype of 95 dogs from 10 different breeds was determined by the “TaqMan” assay method. The IgA genotypes for each dog breed are shown in Table 5. All 10 German Shepherd dogs tested were homozygous for variant C.

TABLE 5
Breed study results using “TaqMan” assay

| | Homozygous | | | | Heterozygous | | | | | |
|---|---|---|---|---|---|---|---|---|---|---|
| Breed | AA | BB | CC | DD | AB | AC | AD | BC | BD | CD |
| German Shepherd | | | 10 | | | | | | | |
| Labrador retriever | | 3 | | 1 | 1 | | 1 | 3 | | |
| Shih Tzu | 7 | 1 | 1 | | | | | | | |
| Rottweiler | 10 | | | | | | | | | |
| Golden retriever | | 4 | 3 | | | | | 2 | | 1 |
| Beagle | | 1 | 1 | 5 | | | | | | 1 |
| Dobermann | 3 | | 7 | | | | | | | |
| Yorkshire Terrier | 4 | 1 | 2 | 1 | | 1 | | 1 | | |
| King Charles | | 5 | | | 5 | | | | | |
| Cavalier Spaniel | | | | | | | | | | |
| West Highland | 4 | | 4 | | 1 | | | | | |
| White Terrier | | | | | | | | | | |

### EXAMPLE 4

The distribution of IgA genotypes in a panel of dietary sensitive dogs was determined. Dietary sensitive dogs are defined as dogs that produce intermittent loose feces, respond to dietary manipulation and whose dietary sensitivity is not food specific. Genotyping was carried out using the “TaqMan” assay as described in Example 3.

### Results

The genotypes of the dietary sensitive panel were compared with the genotypes from the “random” population of dogs from Example 3 (Table 6). The most common genotypes within the panel of dietary sensitive dogs were AC (30%) and BC (35%). A binomial test was carried out to see if there was an association between a particular genotype and dietary sensitivity. Table 7 shows the percentage of dogs in each sample with each allele, and the p-value of the difference between the samples (based on the binomial distribution). Within the panel of dietary sensitive dogs, 80% had one or more variant C allele. In comparison, only 39% of the random population of dogs had one or more variant C allele. The association between dietary sensitivity and the presence of variant C is statistically significant (p=0.00). These results indicate that the presence of one or more variant C allele correlates with susceptibility to non-specific dietary sensitivity.

TABLE 6
Comparison of IgA genotypes

| | | |
|---|---|---|
| Genotype | Random population | Dietary sensitive panel |

| | | | | |
|---|---|---|---|---|
| AA | 28 | (29%) | 2 | (10%) |
| AB | 7 | (7%) | 0 | (0%) |
| AC | 1 | (1%) | 6 | (30%) |
| AD | 1 | (1%) | 0 | (0%) |
| BB | 15 | (16%) | 2 | (10%) |
| BC | 6 | (6%) | 7 | (35%) |
| CC | 28 | (29%) | 2 | (10%) |
| CD | 2 | (2%) | 1 | (5%) |
| DD | 7 | (7%) | 0 | (0%) |
| Total | 95 | | 20 | |

TABLE 7
Frequency of variant alleles

| Allelic variant | Random population | Dietary sensitive panel | P-value |
|---|---|---|---|
| A | 39% | 40% | 0.55 |
| B | 29% | 45% | 0.16 |
| C | 39% | 80% | 0.00 |
| D | 11% | 5% | 0.42 |

### EXAMPLE 5

### Materials and Methods

### Primer and Probe Design

Primers were designed using Primer 3 (Rozen S, Skaletzky H J (2000) Primer3 on the WWW for general users and for biologist programmers. Humana Press, Totowa, N.J.) against the sequences of the four canine IGHA gene variants. Since these genes vary in the coding of the hinge region located at the 5′ end of the second exon, primers were selected that amplified an approximately 160 bp product in all variants encompassing this area of variability (Table 8). The predicted annealing temperature of these primers was 60° C.

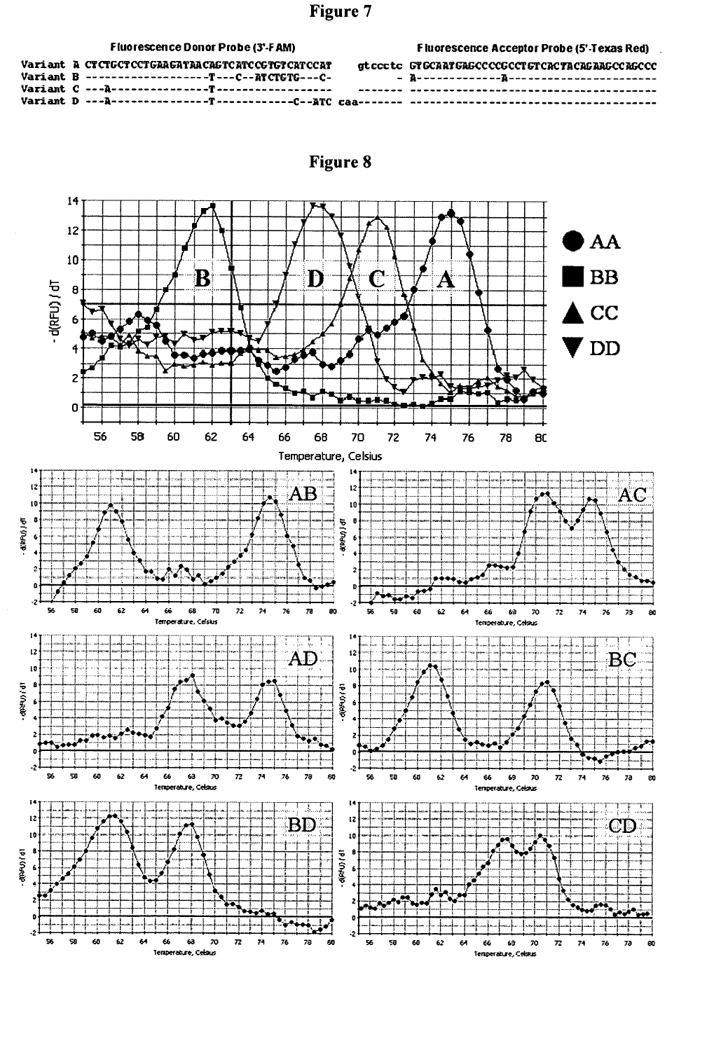

A fluorescence donor probe with a 3′-FAM label was designed to anneal in the hinge region of the four allelic variants of the canine IGHA gene (FIG. 7). This probe was designed as a 100% match to variant A with an annealing temperature of approximately 75° C. The probe had 10, 2 and 6 base mismatches with variants B, C and D respectively. A fluorescence acceptor probe with a 5′-Texas red label was designed to anneal 3′ of the donor probe and was a 100% base match to variants A, C and D and shared two mismatches with variant B (FIG. 7). This probe had a predicted annealing temperature of 85° C. so that this probe would be annealed to its target prior to the donor probe. Due to the different lengths of the hinge regions of the IGHA gene, the base separation between the two fluorescence labelled nucleotides varied from 1 (variant B) to 10 bases (variant D). All primers and probes were synthesised by Eurogentech Ltd. (Romsey, Hampshire, UK). PCR products for direct sequencing were amplified using a primer set used previously for the cloning and sequencing of the IGHA gene variants (see Table 1).

TABLE 8

Primer and Probe Sequences

| | |
|---|---|
| Primers | Probes |

| | | | |
|---|---|---|---|
| 5′ sense | 3′ antisense | Fluorescence donor | Fluorescence acceptor |

| | | | |
|---|---|---|---|
| TGGACACTGA | GGATTGGAGC | CTCTGCTCCTGAAGATAACA | GTGCAATGAGCCCCGCCTGT |
| CCTGTTCCA | CTAAAAGCAG | GTCATCCGTGTCATCCAT | CACTACAGAAGCCAGCCC |

### Samples and DNA Extraction

Surplus EDTA blood was obtained from 96 convenience samples submitted to Langford Veterinary Diagnostics, School of Clinical Veterinary Science, University of Bristol for routine haematological assessment. DNA was extracted from blood using the DNeasy Isolation Kit (Qiagen Ltd., Crawley, U.K.) as per the manufacturer's instructions. Plasmids containing each of the IGHA gene variants were produced as described previously.

### PCR and FRET Analysis

PCR was performed using HotStar-Taq Master Mix (Qiagen Ltd.) using 0.2 μM of each primer and 5 μl of purified DNA in a final volume of 25 μl. Magnesium chloride concentrations were adjusted to 4.5 mM in the final reaction by addition of 50 mM MgCl₂. Sample incubations were performed in a PTC-200 DNA engine (GRI, Braintree, Essex, U.K.) at 95° C. for 15 minutes and then 45 cycles of 95° C. for 10 seconds, 60° C. for 20 seconds and 72° C. for 20 seconds followed by 72° C. for 10 minutes. Following completion of the PCR, the oligonucleotide probes were added to a final concentration of 0.2 μM in a suitable volume of nuclease-free water to give a final volume of 30 μl.

The FRET analysis was performed in an iCycler IQ (Bio-Rad Laboratories Ltd., Hercules, Calif.) by incubating the samples at 95° C. for 1 minute to allow collection of the background fluorescence data from the experimental plate, 50° C. for 1 minute and then a melt curve was produced by heating the samples from 50° C. to 95° C. in 0.5° C. increments with a dwell time at each temperature of 10 s during which the fluorescence data were collected. In order to detect the transfer of energy between the FAM and Texas red fluorophores, the FAM and Texas red emission filters were switched so that the fluorescence data were collected with the FAM excitation and Texas red emission filters. The melting temperatures of the products was determined with the iCycler iQ Optical System Software (version 3: Bio-Rad Laboratories Ltd.) using a rate of change of fluorescence (−d(RFU)/dT) versus temperature graph.

Products for direct sequencing were amplified using the same protocol as for the FRET PCR products. Products were separated using 1% agarose gel electrophoresis and product bands were excised from the gel and then purified using the QIAquick PCR Purification Kit (Qiagen Ltd.) following the manufacturer's instructions. Purified products from three separate reactions were sent to the Sequencing Service (School of Life Sciences, University of Dundee, Dundee, Scotland) for sequencing.

### Results

Plasmids containing each of the IGHA gene variants were used to determine the melting peaks that would be obtained with DNA from dogs homozygous for the four alleles. The FRET experiment was repeated on 10 samples to determine the reproducibility of the melting peaks. The mean temperature (standard deviation) of the melt peaks for variants A, B, C and D were 75.0° C. (0.2), 61° C. (0.4), 70.5° C. (0.3) and 67.5° C. (0.2) respectively. Equimolar mixes of each of the plasmids were combined to produce DNA mixes equivalent to the six possible heterozygous genotypes possible with the four allelic variants (AB, AC, AD, BC, BD and CD). When these mixes were used, two melt peaks were visible at temperatures equivalent to those produced by the appropriate single variant plasmids (FIG. 8).

The FRET probes were then used to genotype DNA obtained from 96 convenience blood samples. The frequency of each allele within the breeds examined was determined from the genotypes obtained from these samples (Table 9). The results indicate that allelic frequency varies between different breeds within the dog population. None of the blood samples examined produced melt peaks that occurred at temperatures different from those recorded for the four variants so far identified. A portion of the IGHA gene was amplified from two dogs with each of the four homozygous genotypes (8 samples in total) and was sent for direct sequencing. The sequence data from these dogs were identical to the variants predicted by the melting peaks found in the FRET genotyping experiments.

TABLE 9
IGHA Gene Allelic Frequencies in Selected Dog Breeds

| | Number | IGHA Allelic Variant | | | |
|---|---|---|---|---|---|
| Breed | n= | A | B | C | D |
| Boxer | 18 | 0.028 | 0 | 0 | 0.972 |
| Cocker Spaniel | 12 | 0.5 | 0.167 | 0.333 | 0 |
| Golden Retriever | 15 | 0.067 | 0.233 | 0.633 | 0.067 |
| Labrador | 20 | 0.025 | 0.425 | 0.55 | 0 |
| Rottweiler | 8 | 1 | 0 | 0 | 0 |
| West Highland White Terrier | 12 | 0.542 | 0.458 | 0 | 0 |
| Crossbred | 11 | 0.273 | 0.273 | 0.364 | 0.09 |
| Total | 96 | 0.266 | 0.234 | 0.297 | 0.203 |

### Discussion

Multiple IgA subclasses have been identified in humans, primates and lagomorphs, whereas in mice, cattle and dogs only a single subclass has previously been characterised. The two human subclasses (IgA₁ and IgA₂) are defined by a difference in the length of the hinge region between the CH₁ and CH₂ domains. The single IgA subclass previously identified in dogs has a hinge region with a predicted amino acid sequence similar to the IgA₁ subclass of humans.

The presence of the extended hinge region of human IgA₁ confers greater flexibility to the immunoglobulin molecule, facilitating antigen binding, but makes it more susceptible to cleavage by protease compared with IgA₂. The shortening of the hinge region that is present in human IgA₂ is due to a 39 base pair deletion from the second exon close to the 5′ end (FIG. 5). This is not caused by a shift in the splice site but is due to a separate gene locus within the immunoglobulin cluster.

Of the four variants identified in canine IgA, variants A, C and D share the greatest sequence similarity, with the difference in mRNA length due to a polymorphism in the splice acceptor. Variant B has the shortest mRNA sequence, similar to variant A, but it also has base deletions towards the 5′ end of the second exon with a greater number of base polymorphisms compared with the other variants. It is therefore possible that variants A, C and D are allelic variants at one gene locus (similar to human IgA₁), and that variant B is encoded within a second locus and may therefore represent a second IgA subclass (similar to IgA₂).

German Shepherd dogs (GSD) are particularly prone to a number of inflammatory and immune-mediated alimentary diseases. All sixteen GSD analysed in a separate study had ‘low’ expression of mRNA when tested with primer set 1, suggesting that none expressed variant A. The susceptibility of GSD to disease and relative IgA deficiency therefore appears to be related to the particular IgA variant(s) that are present within this breed. In the present study, only variant C was found in all of the GSD tested (i.e. all GSD were homozygous for variant C). This suggests that the presence of variant C allele predisposes German Shepherd dogs to gastrointestinal and other IgA-related disorders known to be more prevelant in GSDs than in other breeds. Variant C may also result in a deficiency of IgA, possibly by making the molecule more susceptible to degradation. Furthermore, tests on a panel of non-specific dietary sensitive dogs showed that 80% had one or more variant C allele, in comparison to 39% of dogs in a random population. This suggests that the presence of a variant C allele increases susceptibility to gastrointestinal disease.

A FRET probe based assay was successfully developed to determine the IGHA hinge region genotype of an individual dog. The sequence differences between the allelic variants of the canine IGHA gene involve a number of base polymorphisms, insertions and deletions which makes the development of a single assay to discriminate between all variants difficult as the differences between any two variants are not necessarily in the same region. Previous studies have either utilised two fluorescent probes or a fluorescent probe and an internally labelled primer. The two-probe method was selected for use in this assay, as only deoxyadenosine-linked internal fluorescent dyes were commercially available for primer synthesis. A suitable internally labelled antisense primer with a deoxyadenosine base near the 3′ end which amplified all gene variants could not be successfully designed due to the polymorphisms in the variant B sequence 3′ of the area of interest. Design of a sense internally labelled primer was impossible due to the variable number of deoxycytosine and deoxythymine base repeats present 5′ to the area of interest. Another difficulty in the design of this assay was the length of the base separation between the donor and acceptor fluorophores on the two probes.

Due to the variable length of the hinge region of the IGHA gene in the different alleles, there was a separation of between one and ten bases between the two probes used. The spatial separation of the two fluorophores is important as increasing distance between them leads to a decrease in the efficiency of energy transfer. In order to minimize the fluorophore separation when the probes were hybridized to variant D template, the fluorescence acceptor probe was designed in a region that included two base polymorphisms when compared with variant B DNA. This variant had the greatest number of base mismatches with the donor probe and was likely to have the lowest melting temperature. The reduction in the annealing temperature of the acceptor probe due to these two base mismatches was not expected to affect the detection of this variant. An alternative solution would have been the use of a probe that was a 100% match to the variant B sequence but this would have required two reactions for each sample to allow determination of the complete genotype, whereas the use of a single probe with mismatches required only a single reaction. The present method therefore allows the genotyping of an individual from a single PCR reaction in a relatively short period of time with the analysis completed within approximately 90 minutes.

1. A method for determining susceptibility to an IgA-related disorder in an animal, the method comprising:
a) identifying the or each IgA allelic variant present in a sample from the animal; and
b) thereby determining whether the animal is susceptible to an IgA-related disorder.

2. A method according to claim 1, wherein the animal is a dog.

3. A method according to claim 1, wherein the IgA-related disorder is gastrointestinal, skin, respiratory, rheumatoid or periodontal disease.

4. A method according to claim 3, wherein the disease is diarrhea, small intestinal bacterial overgrowth, inflammatory bowel disease, perianal fistulas, atopic dermatitis, pyoderma, anal furunculosis, malasessia infestans or disseminated aspergillosis.

5. A method according to claim 1, wherein identification of the allelic variant comprises detecting one or more polymorphisms in the hinge region of the IgA allelic variant, or a polymorphism which is in linkage disequilibrium with such a polymorphism.

6. A method according to claim 5, wherein the polymorphism is at any one of the following positions in relation to SEQ ID NO: 1:
position 179 [C/T];
position 370 [T/C];
position 371 [T/C];
position 372 [C/G];
position 375 [G/T];
positions 514 to 546 [number of CT repeats];
position 547 [T/A];
position 563 [A/T];
positions 563 to 571 [deletion];
positions 576 to 578 [addition];
position 582 [C/T];
position 583 [A/G];
position 584 [T/A];
position 592 [G/A]; or
position 606 [G/A];

7. A method according to claim 1, wherein the presence of at least one variant C allele indicates susceptibility to an IgA-related disorder.

8. A method according to claim 1, wherein step (a) comprises contacting a polynucleotide encoding an IgA allelic variant with a specific binding agent for an allelic variant and determining whether the agent binds to the polynucleotide, wherein binding of the agent to the polynucleotide indicates the presence of the allelic variant.

9. A method according to claim 8 wherein the agent is a polynucleotide which is able to bind a polynucleotide encoding the IgA allelic variant but which does not bind a polynucleotide encoding a different IgA allelic variant.

10. A method according to claim 1, wherein step (a) comprises contacting an IgA allelic variant polypeptide with a specific binding agent for an allelic variant and determining whether the agent binds to the polypeptide, wherein binding of the agent to the polypeptide indicates the presence of the allelic variant.

11. A method according to claim 1, wherein the allelic variant is detected by measuring the mobility of an IgA allelic variant polypeptide or a polynucleotide encoding an IgA allelic variant during gel electrophoresis.

12. A method according to claim 1, wherein the allelic variant is detected by means of fluorescence resonance energy transfer.

13. A method according to claim 12, wherein step (a) comprises:
providing a first probe that binds to a conserved sequence adjacent to a region of sequence variation between the allelic variants;
providing a second probe that binds to said region of sequence variation and which has a different melting temperature for each allelic variant sequence, wherein each probe is labelled with a fluorophore that allows fluorescence resonance energy transfer when both probes are bound to the target sequence;
combining said first and second probes with a sample which comprises a polynucleotide comprising the target sequence;
varying the temperature of the sample and detecting the fluorescence emitted; and
determining thereby the melting temperature of the second probe and hence the or each allelic variant present in the sample.

14. A method according to claim 13, wherein the gap between the adjacent labelled ends of the two probes is from 1 to 10 nucleotides when both probes are bound to the target sequence.

15. A method according to claim 13, wherein the second probe binds to the hinge region of an IgA allelic variant.

16. A probe, primer or antibody which is capable of detecting an IgA allelic variant.

17. A probe according to claim 16, which comprises the sequence
GTGCAATGAGCCCCGCCTGTCACTACAGAAGCCAGCCC
or
CTCTGCTCCTGAAGATAACAGTCATCCGTGTCATCCAT.

18. A kit for carrying out the method of claim 1 comprising means for detecting an IgA allelic variant.

19. A kit according to claim 18, comprising a probe, primer or antibody according to claim 16.

20. A method of preparing customized food for an animal which is susceptible to an IgA-related disorder, the method comprising:
(a) determining whether the animal is susceptible to an IgA-related disorder by a method according to claim 1; and
(b) preparing food suitable for the animal.

21. A method according to claim 20, wherein the customized animal food comprises ingredients which prevent or alleviate an IgA-related disorder, and/or does not comprise ingredients which contribute to or aggravate an IgA-related disorder.

22. A method according to claim 21 wherein the customized animal food comprises β-glucans, glutamine, probiotics, oligosaccharides, exogenous IgA, hypoallergenic protein, hydrolysed protein, vitamin C, taurine, curcumin, aloe vera, lutein, lycopene, β-carotene, eicosapentaenoic acid (EPA) or docosahexaenoic acid (DHA).

23. A method according to claim 20, further comprising providing the food to the animal, the animal's owner or the person responsible for feeding the animal.

24. A method of providing a customized animal food, comprising providing food suitable for an animal which is susceptible to an IgA-related disorder to the animal, the animal's owner or the person responsible for feeding the animal, wherein the animal has been genetically determined to be susceptible to an IgA-related disorder.

25. A method for identifying an agent for the treatment of an IgA-related disorder, the method comprising:
(a) contacting an IgA allelic variant polypeptide or a polynucleotide which encodes an IgA allelic variant with a test agent; and
(b) determining whether the agent is capable of binding to the polypeptide or modulating the activity or expression of the polypeptide or polynucleotide.

26. A method of treating an animal for an IgA-related disorder, the method comprising administering to the animal an effective amount of a therapeutic compound which prevents or treats the disorder, wherein the animal has been identified as being susceptible to an IgA-related disorder by a method according to claim 1.

27. A database comprising information relating to IgA allelic variants and optionally their association with IgA-related disorder(s).

28. A method for determining whether an animal is susceptible to an IgA-related disorder, the method comprising:
(a) inputting data of one or more IgA allelic variant(s) present in the animal
to a computer system;
(b) comparing the data to a computer database, which database comprises information relating to IgA allelic variants and the IgA-related disorder susceptibility associated with the variants; and
(c) determining on the basis of the comparison whether the animal is susceptible to an IgA-related disorder.

29. A method according to claim 28, wherein the IgA allelic variant(s) are as defined in claim 6.

30. A computer program comprising program code that, when executed on a computer system, instructs the computer system to perform all the steps of claim 28.

31. A computer system arranged to perform a method according to claim 28 comprising:
(a) means for receiving data of the one or more IgA allelic variant(s) present in the animal;
(b) a module for comparing the data with a database comprising information relating to IgA allelic variants and the IgA-related disorder susceptibility associated with the variants; and
(c) means for determining on the basis of said comparison whether the animal is susceptible to an IgA-related disorder.

32. A method of preparing customized food for an animal which is susceptible to an IgA-related disorder, the method comprising:
(a) determining whether the animal is susceptible to an IgA-related disorder by a method according to claim 28;
(b) electronically generating a customized animal food formulation suitable for the animal;
(c) generating electronic manufacturing instructions to control the operation of food manufacturing apparatus in accordance with the customized animal food formulation; and
(d) manufacturing the customized animal food according to the electronic manufacturing instructions.

33. A computer system according to claim 31, further comprising:
(d) means for electronically generating a customized animal food formulation suitable for the animal;
(e) means for generating electronic manufacturing instructions to control the operation of food manufacturing apparatus in accordance with the customized animal food formulation; and
(f) a food product manufacturing apparatus.

34. An isolated polynucleotide comprising:
(a) an IgA variant sequence that differs to SEQ ID NO: 1 at one or more polymorphic positions as defined in claim 6;
(b) any one of SEQ ID NO:s 3, 5, 35, 7 or 9;
(c) a sequence that is complementary or is degenerate as a result of the genetic code to a sequence as defined in (a) or (b); or
(d) a fragment of (a), (b) or (c) which differs to SEQ ID NO: 1 at one or more polymorphic positions as defined in claim 6 and which is at least 10 nucleotides in length.

35. A polypeptide comprising:
(a) a sequence encoded by a polynucleotide according to claim 38;
(b) any one of SEQ ID NO:s 4, 6, 8 or 10; or
(c) a fragment of (a) or (b) which differs to SEQ ID NO: 2 at one or more polymorphic positions as defined in claim 6 and which is at least 10 amino acids in length.
